SEQUENCE LISTING (31 sequences)

SEQ ID NO: 1 - protein, 3 aa, Artificial
  agonist for formyl peptide receptors
MMK

SEQ ID NO: 2 - protein, 5 aa, Artificial
  agonist for formyl peptide receptors
WKYMV

SEQ ID NO: 3 - protein, 6 aa, Artificial
  agonist for formyl peptide receptors
WKYMVM

SEQ ID NO: 4 - protein, 133 aa, Staphylococcus aureus
MKKNITKTIIASTVIAAGLLTQTNDAKAFFSYEWKGLEIAKNLADQAKKDDERIDKLMKESDKNLTPYKAETVNDLYLIVKKLSQGDVKKAVVRIKDGGPRDYYTFDLTRPLEENRKNIKVVKNGEIDSIYWD

SEQ ID NO: 5 - protein, 132 aa, Staphylococcus aureus
MKKNITKTIIASTVIAAGLLTQTNDAKAFFSYEWKGLEIAKNLADQAKKDDERADKLIKEADEKNEHYKGKTVEDLYVIAKKMGKGNTIAVVKIKDGGKNGYYTFDITRPLEEHRKNIPVVKNGEIDSITWY

SEQ ID NO: 6 - DNA, 20 bp, Artificial
  forward primer FLIPr
ttctttagttatgaatggaa

SEQ ID NO: 7 - DNA, 22 bp, Artificial
  reverse primer FLIPr
ttaatcccaataaatcgagtcg

SEQ ID NO: 8 - DNA, 36 bp, Artificial
  sense primer
gctctagaaataattttgtttaactttaagaaggag

SEQ ID NO: 9 - DNA, 50 bp, Artificial
  antisense primer
tctaaacctttccattcataactaaagaacttgtcgtcatcgtcgtacag

SEQ ID NO: 10 - DNA, 20 bp, Artificial
  sense primer
ttctttagttatgaatggaa

SEQ ID NO: 11 - DNA, 34 bp, Artificial
  antisense primer
cgtcctgaattcttaatcccaataaatcgagtcg

SEQ ID NO: 12 - DNA, 60 bp, Artificial
  FPR sense primer
ccggaattcatggactacaaggacgacgacgacaagatgatggagacaaattcctctctc

SEQ ID NO: 13 - DNA, 29 bp, Artificial
  FPR antisense primer
gctctagatcactttgcctgtaacgccac

SEQ ID NO: 14 - DNA, 61 bp, Artificial
  FPRL1 sense primer
ccggaattcatggactacaaggacgacgacgacaagatggaaaccaacttctccactcctc

SEQ ID NO: 15 - DNA, 28 bp, Artificial
  FPRL1 antisense primer
gctctagatcacattgcctgtaactcag

SEQ ID NO: 16 - DNA, 57 bp, Artificial
  C5aR sense primer
ccggaattcatggactacaaggacgacgacgacaagatgaactccttcaattatacc

SEQ ID NO: 17 - DNA, 28 bp, Artificial
  C5aR antisense primer
gctctagactacactgcctgggtcttct

SEQ ID NO: 18 - protein, 8 aa, Artificial
  N-terminal FLAG-tag
DYKDDDDK

SEQ ID NO: 19 - DNA, 12 bp, Artificial
  sense primer
ttctttagttat

SEQ ID NO: 20 - DNA, 28 bp, Artificial
  reverse primer
gccgaattcttaataccaagtaatcgaa

SEQ ID NO: 21 - protein, 104 aa, Staphylococcus aureus
FFSYEWKGLEIAKNLADQAKKDDERADKLIKEADEKNEHYKGKTVEDLYVIAKKMGKGNTIAVVKIKDGGKNGYYTFDITRPLEEHRKNIPVVKNGEIDSITWY

SEQ ID NO: 22 - DNA, 39 bp, Artificial
  forward primer CHIPS-FLIPr
gtttacttttgaaccgtttaaaggtttagaaatcgcaaa

SEQ ID NO: 23 - DNA, 39 bp, Artificial
  forward primer FLIPr-CHIPS
gttctttagttatgaatggcctacaaatgaagaaataga

SEQ ID NO: 24 - DNA, 26 bp, Artificial
  forward primer FLIPr-D1F
gtttagttatgaatggaaaggtttag

SEQ ID NO: 25 - DNA, 23 bp, Artificial
  forward primer FLIPr-D1F2F
gagttatgaatggaaaggtttag

SEQ ID NO: 26 - DNA, 33 bp, Artificial
  reverse primer CHIPS1-6-FLIPr7-104, FLIPr-D1F and FLIPr-D1F2F
gtcctgaattcttaatcccaataaatcgagtcg

SEQ ID NO: 27 - DNA, 36 bp, Artificial
  reverse primer FLIPr1-6-CHIPS7-121
gctactagctgaattcttagtatgcatattcattag

SEQ ID NO: 28 - protein, 6 aa, Artificial
  CHIPS derived peptide
FTFEPF

SEQ ID NO: 29 - protein, 6 aa, Artificial
  N-terminal substitution of CHIPS derived peptide
FFWYEW

SEQ ID NO: 30 - protein, 105 aa, Staphylococcus aureus
FFSYEWKGLEIAKNLADQAKKDDERIDKLMKESDKNLTPYKAETVNDLYLIVKKLSQGDVKKAVVRIKDGGPRDYYTFDLTRPLEENRKNIKVVKNGEIDSIYWD

SEQ ID NO: 31 - protein, 104 aa, Staphylococcus aureus; identical to SEQ ID NO: 21

(19) United States
(12) Patent Application Publication
(10) Pub. No.: US 2009/0264359 A1
(43) Pub. Date: Oct. 22, 2009

(54) FPLR-1 INHIBITORS FOR USE IN DISEASES INVOLVING AMYLOID-INDUCED INFLAMMATORY EVENTS (FLIPR AND FLIPR-LIKE) AND IMMUNECOMPLEX-MEDIATED DISEASES

(76) Inventors: Cornelis Petrus Maria Van Kessel, Bunnik (NL); Johannes Antonius Gerardus Van Strijp, Odijk (NL)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/298,023

(22) PCT Filed: Jun. 18, 2007
(86) PCT No.: PCT/EP2007/005340

§ 371 Date: Jan. 29, 2009

(30) Foreign Application Priority Data
Jun. 16, 2006 (EP) 06012380.9

(51) Int. Cl. A61K 38/16; C07K 14/00

(52) U.S. Cl. 514/12; 530/324

(57) ABSTRACT

The present invention relates to a FPLR-1 inhibitor selected from the group consisting of FLIPr having the amino acid sequence MKKNITKTIIASTVIAAGLLTQTNDAKAFFSYEWKGLEIAKNLADQAKKDDERIDKLMKESDKNLTPYKAETVNDLYLIVKKLSQGDVKKAVVRIKDGG [symbol] FLIPr-like having the amino acid sequence MKKNITKTIIASTVIAAGLLTQTNDAKAFFSYEWKGLEIAKNLADQAKKDDERADKLIKEADEKNEHYKGKTVEDLYVIAKKMGKGNTIAVVKIKDGGK [symbol] fragments of a) or b) having FPLR-1 inhibitory activity; homologues of a), b) or c) having FPLR-1 inhibitory activity; or derivatives of a), b), c) or d) having FPLR-1 inhibitory activity.

The present invention relates to new staphylococcal anti-inflammatory proteins and biological active fragments thereof. The invention further relates to the use of these proteins and fragments in medicine, in particular in the treatment of diseases involving amyloid-induced inflammatory events or for the treatment of immunecomplex-mediated diseases. The invention also relates to therapeutical compositions comprising such new proteins and fragments.

Staphylococcus aureus remains a leading cause of both community-acquired and hospital-acquired infections. Although S. aureus is a normal commensal of the human skin it can potentially infect any tissue of the body and occasionally spreads from the primary site of infection to cause life threatening diseases like osteomyelitis, endocarditis, pneumonia, and septicemia. Serious S. aureus infection is most often associated with predisposing conditions like chronic illness, traumatic injury including surgery and transcutaneous devices, burns, compromised immune system or other infections.

Bacteria have developed mechanisms to escape the first line of host defense, which is constituted by the recruitment of phagocytes to the sites of bacterial invasion. The ability of S. aureus to cause such a wide range of infections is also the result of its extensive arsenal of virulence factors. Both bacterial surface components and secreted extracellular proteins have been described to contribute to the pathogenesis of infection.

In addition, S. aureus uses efficient strategies to evade recognition by the innate immune system. Nevertheless, the precise role of several individual staphylococcal factors in the development of infection is often difficult to assess and less is known about their interaction with host factors.

Mobilization of phagocytes in response to chemoattractants constitutes the first line of defense against S. aureus infection. Chemoattractants are grouped in the superfamily of chemokines and the “classical” chemoattractants, which include the formylated peptides (side products of bacterial translation), activated complement component 5 (C5a) and C3 (C3a), leukotriene B4 (LTB4), and platelet-activating factor (PAF).

Both classical chemoattractants and chemokines activate seven-transmembrane G protein-coupled receptors (GPCRs) expressed on cells of hematopoietic origin but also on many other cell types.

Chemotaxis Inhibitory protein of S. aureus (CHIPS) was recently described as an excreted protein that impairs the response of neutrophils and monocytes to C5a and formylated peptides such as N-formyl-methionyl-leucyl-phenylalanine (fMLP). CHIPS binds directly to the C5a receptor (C5aR) and formyl peptide receptor (FPR) preventing the natural ligands from activating these receptors.

FPR is the high affinity receptor for fMLP that is activated by picomolar to nanomolar concentrations of fMLP and is expressed on phagocytic leukocytes but also on cell types as diverse as hepatocytes, dendritic cells, astrocytes, and microglia cells. Two other homologs of FPR have been identified, formyl peptide receptor-like1 (FPRL1), and the monocyte- and dendritic cell-expressed formyl peptide receptor-like2 (FPRL2). FPRL1 is considered a low-affinity fMLP receptor and is expressed in an even greater variety of cell types. In the last years, a wide variety of agonists for this receptor has been identified, including components from microorganisms and host-derived peptide and lipid agonists.

It is remarkable that the FPRL1 is used by at least three amyloidogenic ligands, the serum amyloid A (SAA), the 42 amino acid form of β amyloid (Aβ1-42 or Aβ42) and the prion protein fragment PrP106-126. These ligands have been shown to attract phagocytes with important implications in pathological states such as systemic amyloidosis, Alzheimer's disease and prion disease, respectively. FPRL1 has been implicated in different stages of innate immunity by mediating the responses to the antimicrobial peptide LL-37, the acute phase protein serum amyloid A and the endogenous anti-inflammatory lipid mediator lipoxin A4. FPRL1 not only plays a role in innate immune mechanisms but there is also increasing evidence for its implication in the pathogenesis of amyloidogenic diseases. FPRL1 has been reported to mediate the migration and activation of monocytes and microglia induced by Aβ42, participating in Aβ42 uptake and the resultant fibrillar formation. Persistent exposure of macrophages to Aβ42 resulted in retention of Aβ42/FPRL1 complexes in the cytoplasmic compartment and the formation of Congo red positive fibrils.

The pathologic isoform of the prion protein has also been proposed as a chemotactic agonist for the FPRL1. Agents that are able to disrupt the interaction of these components with its receptor may have promising therapeutic potential for FPRL1-mediated diseases.

A few small synthetic peptides such as MMK-1, WKYMVm and WKYMVM, selected from random peptide libraries, have also been identified as agonists for the formyl peptide receptors and are widely used for research purposes. Recently F2L, an acetylated peptide derived from the human heme-binding protein, was identified as a new natural chemoattractant agonist specific for FPRL2.

In the research that led to the invention excreted proteins homologous to CHIPS in the genome of S. aureus were investigated. A gene was found that showed 49% homology with the gene for CHIPS (chp) and contained a leader peptide and a peptidase cleavage site (amino acid sequence AXA). The gene codes for a cleaved 105 amino acid protein with 28% homology with CHIPS:

MKKNITKTIIASTVIAAGLLTQTNDAKAFFSYEWKGLEIAKNLADQAKKD
DERIDKLMKESDKNLTPYKAETVNDLYLIVKKLSQGDVKKAVVRIKDGGP
RDYYTFDLTRPLEENRKNIKVVKNGEIDSIYWD

In this sequence the 105 amino acids that constitute FLIPr are in bold, the signal-peptidase site is underlined. The rest is the signal peptide.

Initial functional assays with the recombinant protein demonstrated a weaker but consistent inhibition of fMLP-induced activation of neutrophils. Further analysis demonstrated that this new protein impairs the neutrophil and monocyte responses to FPRL-1 agonists.

The invention thus relates to a new protein from S. aureus with anti-inflammatory properties: FPRL1 Inhibitory Protein (FLIPr). It is shown herein that FLIPr inhibits the leukocyte response to FPRL1 agonists and binding of FLIPr to HEK293 cells expressing the FPRL1 is demonstrated.

FPRL1 inhibitory protein (FLIPr) inhibits the calcium mobilization in neutrophils stimulated with MMK-1, WKYMVM, prion-protein fragment PrP106-126 and amyloid beta¹⁻⁴² (Aβ1-42). Stimulation with low concentrations of fMLP is partly inhibited. Directed migration is also completely prevented towards MMK-1 and partly towards fMLP.

Fluorescence-labeled FLIPr efficiently binds to neutrophils, monocytes, B-cells and NK-cells. HEK293 cells transfected with human C5aR, FPR, FPRL1 and FPRL2 clearly show that FLIPr directly binds to FPRL1 and, at higher concentrations, also to FPR but not to C5aR and FPRL2.

FLIPr can be used to reveal unknown inflammatory ligands crucial during Staphylococcus aureus infections. This novel FPRL1 antagonist can further be used for the development of therapeutic agents in FPRL1-mediated inflammatory components of diseases such as systemic amyloidosis, Alzheimer and prion disease.

Formyl Peptide Receptor-like 1 Inhibitory Protein (FLIPr) is thus a new staphylococcal anti-inflammatory protein, which constitutes a novel immune evasion mechanism. FLIPr binds directly to the G-protein coupled receptor FPRL1. Because of the importance of FLIPr as a potential anti-inflammatory agent the inventors searched for homologous proteins in the S. aureus genome, as well as its cloning and expression. Simultaneously, recombinant deletion and substitution mutants of FLIPr were constructed to elucidate the active site within the molecule.

The program blasp and the S. aureus genome database at www.ncbi.nlm.nih.gov were used to check for sequence similarities with FLIPr (without the signal peptide). A protein was found showing 73% homology with FLIPr, and was present in two of the six strains screened: hypothetical protein MW1038 (Staphylococcus aureus subsp. aureus MW2) and hypothetical protein SAS1089 (Staphylococcus aureus subsp. aureus MSSA476). The protein, which was named FLIPr-like, contains 104 amino acids (in bold), preceded by a signal peptide and a signal-peptidase site (underlined)

MKKNITKTIIASTVIAAGLLTQTNDAKAFFSYEWKGLEIAKNLADQAKKD
DERADKLIKEADEKNEHYKGKTVEDLYVIAKKMGKGNTIAVVKIKDGGKN
GYYTFDITRPLEEHRKNIPVVKNGEIDSITWY.

FLIPr-like has the same action as FLIPr and binds to FPRL1 and blocks FPRL1-mediated responses, but it is more potent in inhibiting fMLP-induced responses. Furthermore, the existence of two possible active sites within the molecule is shown.

The present invention therefore relates according to a further aspect thereof to the FLIPr-like protein, which is characterized by the above amino acid, and to biologically active fragments thereof.

Part of the immune system is the generation of specific immunoglobulins (especially IgG) that interact with cellular receptors that lead to divergent signals. These receptors are key players in both the afferent and efferent phase of an immune response. Coupling activating receptors with an inhibitory counterpart, discrete thresholds are established that control the window of responses. The specificity of the antibody response is coupled to the innate immune pathways such as complement activation and activation of phagocytes leading to clearance of invading microbes.

Human phagocytes bear activating and inhibitory Fcγ-Receptors, which transmit their signals via immunoreceptor tyrosine-based activation (ITAM) or inhibitory motifs (ITIM) respectively. Four different classes of Fc receptors have been defined: FcγRI (CD64), FcγRII (CD32), FcγRIII (CDl6) and FcγRIV. These Fc receptors display different affinities for the Fc region of IgG. The FcγRII and FcγRIII are the low affinity receptors and the FcγRI the high affinity receptor.

The Fc receptors show significant differences in their affinity for individual antibody isotypes. These differences in affinities for Fc region and isotypes represent checkpoints for the regulation of the immune response. They are important for understanding Fc-receptor-dependent antibody mediated effector functions in vivo and for the possible intervention or therapies.

The inhibitory FcγRIIB is expressed on all cells of the immune system (except T cells and NK cells). It is the only antibody binding Fc receptor on B cells and plays a role in regulating B cell Receptor signals involved in maintaining tolerance and initiation of severe autoreactive processes.

Neutrophils, monocytes and macrophages also coexpress the FcγRIIB with activating Fc receptors and negatively regulate activating signals derived by these receptors. It plays a role in immune complex-mediated inflammation and phagocytosis. Several models in animals deficient in this receptor show an enhancement in Arthus reaction, systemic IgG- and IgE-induced anaphylaxis, anti-GBM glomerulonephritis, immunothrombocytopenia (ITP), haemolytic anemia, collagen-induced arthritis, and IgG-mediated clearance of pathogens and tumors.

The activating Fc receptors signal via an accessory chain, the common γ chain, that carries an ITAM motif required for triggering cell activation. Deletion of this receptor sub-unit leads to functional loss of all activating Fc receptors. In vivo the IgG1 isotype is consistently assigned to the low-affinity receptor FcγRIII. Hence, the most potent antibody isotypes IgG2a and IgG2b are involved in the host response to viral and bacterial infections.

Recently, the mouse FcγRIV is identified with intermediate affinity and restricted subclass specificity, expressed on neutrophils, monocytes, macrophages and dentritic cells. The related protein in humans is FcγRIIIA. The mouse FcγRIV is not expressed on NK cells, while human NK cells express FcγRIIIA. Human neutrophils do not express FcRIIIA, but rather FcγRIIA as their dominant activating FcγR.

The FcγRIIIB is a low affinity receptor expressed on neutrophils that is linked to the plasma membrane via an easily cleaved glycosyl phosphatidylinositol (GPI) anchor. It has been suggested that this receptor plays an important role in the activation of secretory processes and less in phagocytosis.

Other immunoglobulin classes associate with their specific Fc receptor that are structurally related and belong to the immunoglobulin gene superfamily. Each comprises a unique ligand-binding chain which is complexed with the common γ-chain. For IgE, the FceRI is characterized by the markedly high affinity. The low-affinity IgE receptor FceRII (CD23) is structurally unrelated. The FcαRI (CD89) is the only well characterized IgA Fc receptor and is a more distantly related member. The FcαRI is expressed on neutrophils, monocytes, macrophages, eosinophils and some dendritic cells.

Atomic-level structural data are available for FcγRII, FcγRIIb, FcγRIIIb, FcεR1 and FcαRI. The extracellular regions share the same overall heart-shaped structure. The structures are so similar that they can be superimposed. Despite basic sequence similarity for FcαRI, the IgA receptor turns out to have a markedly different structure.

A number of Fc receptor relatives have been recognized recently with potential immunoregulatory capacity in innate and adaptive immune responses. Six human Fc receptor homologs (FcRH1-6), which belong to a conserved gene family, have variable numbers of extracellular immunoglobulin domains and possess cytoplasmic tails with inhibitory motifs. All except FcRH6 are expressed on B cells at different stages in differentiation. The FcRH family remain orphan receptors despite suggestive clues of Fc-binding potential. Stable transfectants failed to demonstrate specific immunoglobulin binding.

The MHC Class-I-related neonatal Fc receptor FcRn is present in epithelial cells, placental syncytiotrophoblasts, as well as endothelial cells and has been implicated in transport of IgG across mucosal cells. Recently, FcRn is shown to be expressed within azurophilic and specific granules of neutrophils and relocates to phagolysosomes on phagocytosis of IgG-opsonized bacteria.

In humans, genetically determined polymorphism exists that involve changes in the extracellular domains affecting ligand binding affinity. For FcγRIIA was shown to have two allelic forms: high and low responder. The HR allotype or R134 (arginine) has low affinity for all human IgG subclasses, particularly IgG2. The LR allotype or H134 (histidine) binds to IgG2 and IgG3 with higher affinity. FcγRIIIA has two allelic forms differing at position 158. The V158 (valine) variant has higher affinity for IgG1, IgG3 and IgG4 than the F158 (phenylalanine) type. For the FcγRIIIB three alleles are recognized: NA1, NA2 and SH. The NA1 type accounts for more efficient phagocytosis of IgG1 and IgG3 opsonized particles.

Fc Receptor polymorphism affects the extracellular ligand-binding domains and therefore plays a role in pathological conditions that involve IgG-FcγR interactions.

In addition, it was found according to the invention that FLIPr and FLIPr-like also inhibit the Fc receptor.

Fc receptors are found on particular cells of the immune system, including phagocytes like macrophages and monocytes, granulocytes like neutrophils and eosinophils, and lymphocytes of the innate immune system (natural killer cells) or adaptive immune system (e.g. B cells). Fc receptors allow these cells to bind to antibodies that are attached to the surface of microbes or microbe infected cells, helping these cells to identify and eliminate microbial pathogens. The Fc receptors bind the antibodies at their Fc region (or tail), an interaction that activates the cell that possesses the Fc receptor.

Immune complexes are clusters of interlocking antigens and antibodies. Under normal conditions immune complexes are rapidly removed from the bloodstream by macrophages in the spleen and Kupffer cells in the liver. In some circumstances, however, immune complexes continue to circulate. Eventually they become trapped in the tissues of the kidneys, lung, skin, joints, or blood vessels. There they set off reactions that lead to inflammation and tissue damage. The pathogenic effects of immune complexes are inter alia induced by interaction with Fc receptors.

According to the invention FLIPr and FLIPr-like and biologically active fragments thereof may thus be used for inhibiting the Fc receptor. In particular, these molecules may be used in the treatment of disorders that involve immune-complex mediated diseases, in particular autoimmune diseases. Examples of conditions that can be treated with FLIPr and FLIPr-like and biologically active fragments thereof are systemic lupus erythematosus (the prototype of systemic autoimmune diseases characterized by autoantibodies to nuclear constituents), rheumatoid arthritis (autoantibodies to the Fc region), idiopathic thrombocytic purpura (autoantibodies to thrombocytes), thrombocytopenia (antibodies for heparin and platelet factor 4), Wegener's granulomatosis (anti-neutrophil cytoplasmic antibodies), myasthenia gravis (autoantibodies acetylcholine receptor), and demyelinating diseases including multiple sclerosis and Guillain-Barre syndrome.

The invention further relates to a therapeutic composition, comprising a suitable excipient, diluent or carrier and FLIPr and/or FLIPr-like protein and/or biologically active fragments thereof for use in the treatment of inflammatory diseases and immune complex-mediated diseases, in particular in the indications described above.

The invention also relates to the use of FLIPr and/or FLIPr-like proteins and/or biologically active fragments thereof for the manufacture of a therapeutic preparation for the treatment of inflammatory diseases and immune complex-mediated diseases, in particular in the indications described above.

The therapeutic compositions, which according to the invention contain FLIPr or FLIPr-like proteins or biologically active as active ingredient, are particularly intended for parenteral, and then specifically, intravenous use. The therapeutic compositions can be prepared by combining (i.e. mixing, dissolving etc.) FLIPr and/or FLIPr-like and/or biologically active fragments of these with pharmaceutically acceptable excipients for intravenous administration. The concentration of the active ingredient in a therapeutic composition can vary between 0.001% and 100%, depending on the nature of the treatment and the method of administration. The dose of the active ingredient for administering likewise depends on the administering route and application, but may for instance vary between 0.001 and 1 mg per kg of body weight, preferably between 1 g and 100 g per kg of body weight.

According to the invention also homologues of FLIPr or FLIPr-like and derivatives thereof can be used. Such homologues or derivatives must be functional. Derivatives may for example be fragments, such as peptides, truncated proteins, chimeric proteins comprising at least a functional part of FLIPr or FLIPr-like and another part, or peptidomimetic versions of the protein.

More specifically derivatives comprise polypeptides or peptides that comprise fewer amino acids than the full length FLIPr or FLIPr-like but still inhibit FPLR-1 and/or the Fc receptor. Such derivatives preferably comprise a stretch of consecutive amino acids but combinations of active domains, optionally spaced by linkers, are also possible. The skilled person is very well capable of defining such derivatives on the basis of the FLIPr or FLIPr-like sequences given herein and testing the thus defined derivative for the required activity as described in the Examples.

In some cases the potential for use of (poly)peptides in drugs may be limited for several reasons. In particular peptides may for example be too hydrophilic to pass membranes like the cell-membrane and the blood-brain barrier, and may be rapidly excreted from the body by the kidneys and the liver, resulting in a low bioavailability. Furthermore, they may suffer from a poor biostability and chemical stability since they may be quickly degraded by proteases, e.g. in the gastro-intestinal tract. Also, peptides generally are flexible compounds which can assume thousands of conformations. The bioactive conformation usually is only one of these possibilities, which sometimes might lead to a poor selectivity and affinity for the target receptor. Finally, the potency of the peptides may not be sufficient for therapeutical purposes.

As a result of the above described drawbacks, (poly)peptides are sometimes mainly used as sources for designing other drugs, and not as actual drugs themselves. In such case it is desirable to develop compounds in which these drawbacks have been reduced. Alternatives for peptides are the so-called peptidomimetics. Peptidomimetics based on FLIPr or FLIPr-like are also part of this application. In that case, one or more of the amino acids in FLIPr or FLIPr-like or a derivative thereof are substituted with peptidomimetic building blocks.

In general, peptidomimetics can be classified into two categories. The first consists of compounds with non-peptidelike structures, often scaffolds onto which pharmacophoric groups have been attached. Thus, they are low molecular-weight compounds and bear no structural resemblance to the native peptides, resulting in an increased stability towards proteolytic enzymes.

The second main class of peptidomimetics consists of compounds of a modular construction comparable to that of peptides, i.e. oligomeric peptidomimetics. These compounds can be obtained by modification of either the peptide side chains or the peptide backbone. Peptidomimetics of the latter category can be considered to be derived of peptides by replacement of the amide bond with other moieties. As a result, the compounds are expected to be less sensitive to degradation by proteases. Modification of the amide bond also influences other characteristics such as lipophilicity, hydrogen bonding capacity and conformational flexibility, which in favourable cases may result in an overall improved pharmacological and/or pharmaceutical profile of the compound.

Oligomeric peptidomimetics can in principle be prepared starting from monomeric building blocks in repeating cycles of reaction steps. Therefore, these compounds may be suitable for automated synthesis analogous to the well-established preparation of peptides in peptide synthesizers. Another application of the monomeric building blocks lies in the preparation of peptide/peptidomimetic hybrids, combining natural amino acids and peptidomimetic building blocks to give products in which only some of the amide bonds have been replaced. This may result in compounds which differ sufficiently from the native peptide to obtain an increased biostability, but still possess enough resemblance to the original structure to retain the biological activity.

Suitable peptidomimetic building blocks for use in the invention are amide bond surrogates, such as the oligo-β-peptides (Juaristi, E. Enantioselective Synthesis of b-Amino Acids; Wiley-VCH: New York, 1996), vinylogous peptides (Hagihari, M. et al., J. Am. Chem. Soc. 1992, 114, 10672-10674), peptoids (Simon, R. J. et al., Proc. Natl. Acad. Sci. USA 1992, 89, 9367-9371; Zuckermann, R. N. et al., J. Med. Chem. 1994, 37, 2678-2685; Kruijtzer, J. A. W. & Liskamp, R. M. J. Tetrahedron Lett. 1995, 36, 6969-6972); Kruijtzer, J. A. W. Thesis; Utrecht University, 1996; Kruijtzer, J. A. W. et al., Chem. Eur. J. 1998, 4, 1570-1580), oligosulfones (Sommerfield, T. & Seebach, D. Angew. Chem., Int. Ed. Eng. 1995, 34, 553-554), phosphodiesters (Lin, P. S.; Ganesan, A. Bioorg. Med. Chem. Lett. 1998, 8, 511-514), oligosulfonamides (Moree, W. J. et al., Tetrahedron Lett. 1991, 32, 409-412; Moree, W. J. et al., Tetrahedron Lett. 1992, 33, 6389-6392; Moree, W. J. et al., Tetrahedron 1993, 49, 1133-1150; Moree, W. J. Thesis; Leiden University, 1994; Moree, W. J. et al., J. Org. Chem. 1995, 60, 5157-5169; de Bont, D. B. A. et al., Bioorg. Med. Chem. Lett. 1996, 6, 3035-3040; de Bont, D. B. A. et al., Bioorg. Med. Chem. 1996, 4, 667-672; Löwik, D. W. P. M. Thesis; Utrecht University, 1998), peptoid sulfonamides (van Ameijde, J. & Liskamp, R. M. J. Tetrahedron Lett. 2000, 41, 1103-1106), vinylogous sulfonamides (Gennari, C. et al., Eur. J. Org. Chem. 1998, 2437-2449), azatides (or hydrazinopeptides) (Han, H. & Janda, K. D. J. Am. Chem. Soc. 1996, 118, 2539-2544), oligocarbamates (Paikoff, S. J. et al., Tetrahedron Lett. 1996, 37, 5653-5656; Cho, C. Y. et al., Science 1993, 261, 1303-1305), ureapeptoids (Kruijtzer, J. A. W. et al., Tetrahedron Lett. 1997, 38, 5335-5338; Wilson, M. E. & Nowick, J. S. Tetrahedron Lett. 1998, 39, 6613-6616) and oligopyrrolinones (Smith III, A. B. et al., J. Am. Chem. Soc. 1992, 114, 10672-10674).

The vinylogous peptides and oligopyrrolinones have been developed in order to be able to form secondary structures (β-strand conformations) similar to those of peptides, or mimic secondary structures of peptides. All these oligomeric peptidomimetics are expected to be resistant to proteases and can be assembled in high-yielding coupling reactions from optically active monomers (except the peptoids).

Peptidosulfonamides are composed of α- or β-substituted amino ethane sulfonamides containing one or more sulfonamide transition-state isosteres, as an analog of the hydrolysis of the amide bond. Peptide analogs containing a transition-state analog of the hydrolysis of the amide bond have found a widespread use in the development of protease inhibitor.

Another approach to develop oligomeric peptidomimetics is to completely modify the peptide backbone by replacement of all amide bonds by nonhydrolyzable surrogates e.g. carbamate, sulfone, urea and sulfonamide groups. Such oligomeric peptidomimetics may have an increased metabolic stability. Recently, an amide-based alternative oligomeric peptidomimetics has been designed viz. N-substituted Glycine-oligopeptides, the so-called peptoids. Peptoids are characterized by the presence of the amino acid side chain on the amide nitrogen as opposed to being present on the α-C-atom in a peptide, which leads to an increased metabolic stability, as well as removal of the backbone chirality. The absence of the chiral α-C atom can be considered as an advantage because spatial restrictions which are present in peptides do not exist when dealing with peptoids. Furthermore, the space between the side chain and the carbonyl group in a peptoid is identical to that in a peptide. Despite the differences between peptides and peptoids, they have been shown to give rise to biologically active compounds.

Translation of a peptide chain into a peptoid peptidomimetic may result in either a peptoid (direct-translation) or a retropeptoid (retro-sequence). In the latter category the relative orientation of the carbonyl groups to the side chains is maintained leading to a better resemblance to the parent peptide.

Review articles about peptidomimetics that are incorporated herein by reference are:

Adang, A. E. P. et al.; Recl. Trav. Chim. Pays-Bas 1994, 113, 63-78; Giannis, A. & Kolter, T. Angew. Chem. Int. Ed. Engl. 1993, 32, 1244-1267; Moos, W. H. et al., Annu. Rep. Med. Chem. 1993, 28, 315-324; Gallop, M. A. et al., J. Med. Chem. 1994, 37, 1233-1251; Olson, G. L. et al., J. Med. Chem. 1993, 36, 3039-30304; Liskamp, R. M. J. Recl. Trav. Chim. Pays-Bas 1994, 113, 1-19; Liskamp, R. M. J. Angew. Chem. Int. Ed. Engl. 1994, 33, 305-307; Gante, J. Angew. Chem. Int. Ed. Engl. 1994, 33, 1699-1720; Gordon, E. M. et al., Med. Chem. 1994, 37, 1385-1401; and Liskamp, R. M. J. Angew. Chem. Int. Ed. Engl. 1994, 33, 633-636.

The invention thus furthermore relates to molecules that are not (poly)peptides themselves but have a structure and function similar to those of FLIPr or FLIPr-like or derivatives thereof.

As used herein the term “biologically active fragments” is intended to encompass besides actual fragments, that have an amino acid sequence that is shorter that the native FLIPr and FLIPr-like, also derivatives and homologues as described above that perform the same function and are also antagonists of FPLR-1 and of the Fc receptor.

The invention will be further elucidated with reference to the Examples that follow and that are not intended to be limiting. In the Examples reference is made to the following figures.

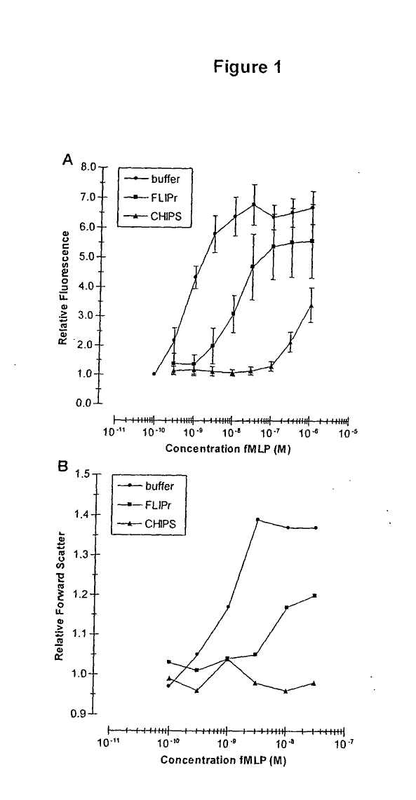

FIG. 1. FLIPr inhibits fMLP-induced calcium mobilization and change in forward scatter of neutrophils. Neutrophils were incubated with buffer (), 3 μg/ml FLIPr (▪) or CHIPS (▴) for 20 minutes at room temperature. (A) For calcium mobilization cells were preloaded with Fluo-3. Each sample was first measured for about 10 seconds to determine the basal fluorescence and subsequently fMLP (concentrations from 10⁻⁶ to 10⁻¹⁰ M) was added and rapidly placed back in the sample holder to continue the measurement. Cells were analyzed in a flow cytometer and activation was expressed as the ratio of the fluorescence value before (cells acquired between T=5 till 7 seconds)/after addition of stimulus (cells acquired at T=12 till 14 seconds after stimulation). Data are mean±SEM of three independent experiments. (B) Neutrophils were challenged with different concentrations fMLP for 15 min at 37° C., fixed with 1% paraformaldehyde and analyzed in a flow cytometer. The relative change in forward scatter value as compared to control cells incubated in buffer only was determined. A representative experiment is shown.

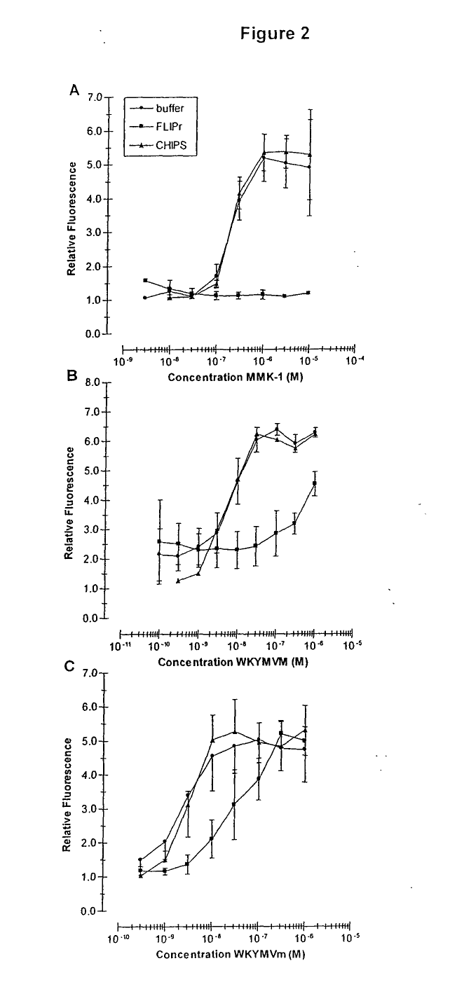

FIG. 2. FLIPr inhibits FPRL1 agonist-induced calcium mobilization in neutrophils. The activity of FLIPr was tested in calcium mobilization assays with neutrophils in response to synthetic peptide FPRL1 agonists MMK-1 (A), WKYMVM (B) and WKYMVm (C). Fluo-3-loaded neutrophils were incubated with buffer (), 3 μg/ml FLIPr (▪) or CHIPS (▴) for 20 minutes. Data are mean±SEM of three independent experiments.

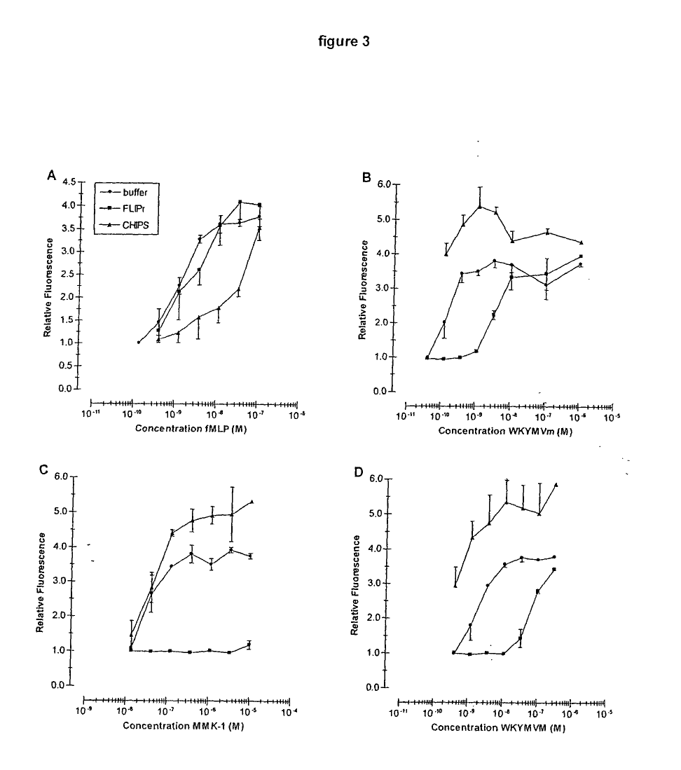

FIG. 3. FLIPr inhibits FPRL1 agonist-induced calcium mobilization in monocytes. The activity of FLIPr was tested in calcium mobilization assays with PBMC in response to the following synthetic peptides: fMLP (A), WKYMVm (B), MMK-1 (C) and WKYMVM (D). Fluo-3-loaded PBMC were incubated with buffer (), 3 μg/ml FLIPr (▪) or CHIPS (▴) for 20 minutes. Monocytes were gated based on scatter parameters and anti-CD14-PE staining. Data are mean±SEM of three independent experiments.

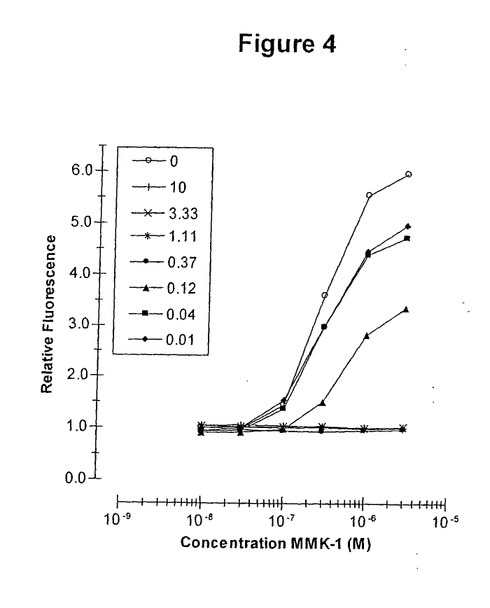

FIG. 4. Potency of FLIPr to inhibit the MMK-1-induced calcium mobilization in neutrophils. The activity of different concentrations FLIPr was tested in calcium mobilization assays with neutrophils in response to synthetic peptide FPRL1 agonist MMK-1. A representative experiment is shown.

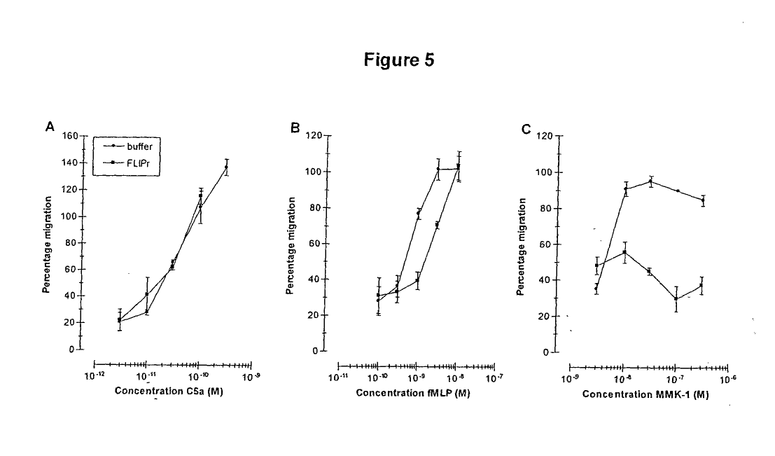

FIG. 5. FLIPr inhibits chemotaxis of neutrophils to fMLP and MMK-1 and not to C5a. Chemotaxis of human neutrophils towards several chemoattractants was measured in a multiwell trans-membrane system. Cells were loaded with Calcein and incubated with buffer () or 3 μg/ml of FLIPr (▪). Dilutions of the chemoattractants C5a (A), fMLP (B) and MMK-1 (C) were placed into each well in triplicate and, after assembling the membrane holder, labeled cells were added to each upper well. The plate was incubated for 30 minutes at 37° C.+5% CO₂, and after washing the membrane holder, fluorescence was measured. Results are expressed as percentage of chemotaxis, and data are mean±SEM of triplicates from one representative experiment out of three. Spontaneous migration towards buffer loaded wells was 29%.

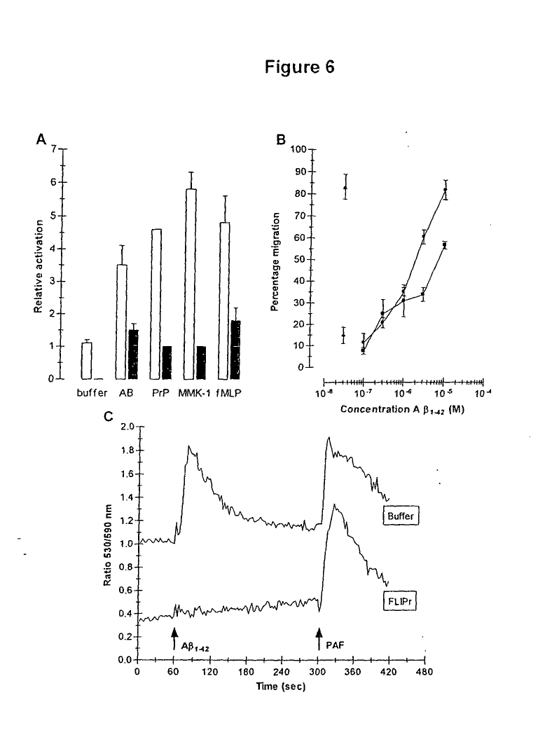

FIG. 6. FLIPr inhibits chemotaxis and calcium flux in response to the endogenous peptide agonist Aβ1-42 and PrP106-126. The activity of FLIPr to inhibit the neutrophil response to FPRL1-endogenous agonists Aβ1-42 and PrP106-126 was tested by chemotaxis and calcium mobilization. (A) The calcium flux induced by 10 μM Aβ1-42 (AB) and 50 μM PrP106-126 (PrP) were inhibited by 3 μg/ml FLIPr. In the same experiment the peptide agonists MMK-1 (1×10⁻⁷M) and fMLP (1×10⁻⁹M) were included. Open bars represent the response of buffer control cells and solid bars the response in the presence of FLIPr. (B) Chemotaxis results towards different concentrations Aβ1-42 of control cells () and cells incubated with 3 μg/ml FLIPr (▪). Data are expressed as percentage migration and are mean±SEM of triplicates of one representative experiment. Controls are included of chemotaxis in response to 3×10⁻⁷M MMK-1 in control cells (♦) and in cells incubated with FLIPr (▴). Spontaneous migration towards buffer was 21.8%. (C) Representative experiments showing Aβ1-42 (10⁻⁵M at 60 seconds) induced calcium mobilization in Fura-2 loaded neutrophils treated with buffer, or 3 μg/ml FLIPr. The same cells were rechallenged at 300 seconds with 10⁻⁹M PAF. Results are depicted as the ratio of the fluorescence at 530/590 nm and shifted to show the individual curves.

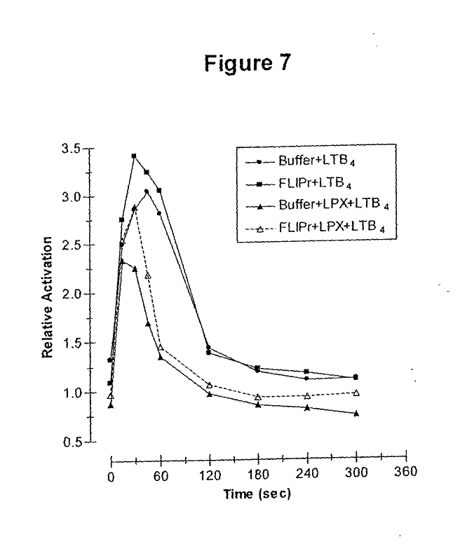

FIG. 7. FLIPr does not interfere with lipoxin A4-mediated FPRL1 activation. The leukotriene B4-induced (LTB4; 10⁻⁹M) actin polymerization is partly prevented by the incubation of neutrophils with 10⁻⁶M Lipoxin A4. Preincubation of neutrophils with 3 μg/ml FLIPr did not interfere with the LTB4-induced response nor the lipoxin-A4 response. Actin polymerization was determined at 15 second intervals with Alexa-labeled Phallacidin and flow cytometry for cells plus LTB4 (), FLIPr and LTB4 (▪), Lipoxin-A4 and LTB4 (▴), and FLIPr+lipoxin-A4 and LTB4 (dashed line, Δ). Results are expressed as the relative increase in fluorescence compared to non-stimulated cells (mean of two representative experiments).

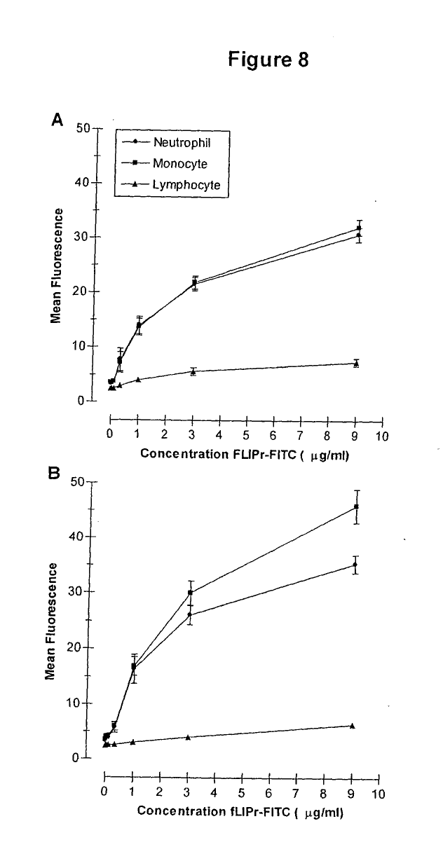

FIG. 8. FLIPr binds to neutrophils, monocytes and a proportion of lymphocytes. Isolated PMN and PBMC were incubated with a range of concentrations of FLIPr-FITC (0.03 to 9 mg/ml) for 30 minutes on ice (A) or at 37° C. (B) under constant shaking. Cells were then washed and resuspended in RPMI-HSA and fluorescence was measured in a flow cytometer. Cells were identified based on scatter parameters and anti-CD14 staining; neutrophils (e), monocytes (▪) and lymphocytes (▴) are displayed. Data are mean±SEM of three independent experiments.

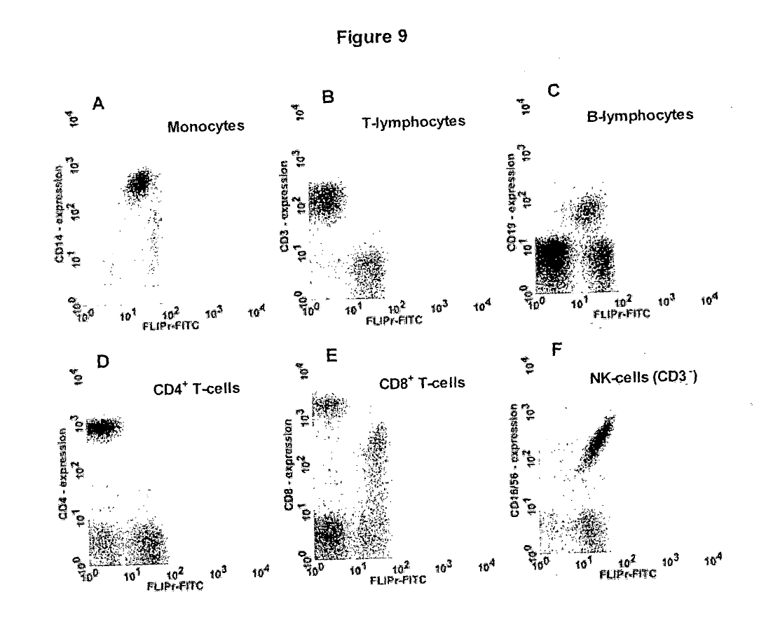

FIG. 9. FLIPr binds to different subsets of leukocytes. Monoclonal antibodies for different subsets of mononuclear cells were used to check the binding profile of FLIPr-FITC by flow cytometry. FLIPr binds to CD14+ monocytes (A); not to CD3+ lymphocytes (T-cells) (B); binds to CD19+ lymphocytes (B-cells) (C); not to CD4+ T-cells (D); binds to a subpopulation of CD8+ T-cells (E), and to CD3−/CD56+/CD16+ lymphocytes (NK-cells) (F).

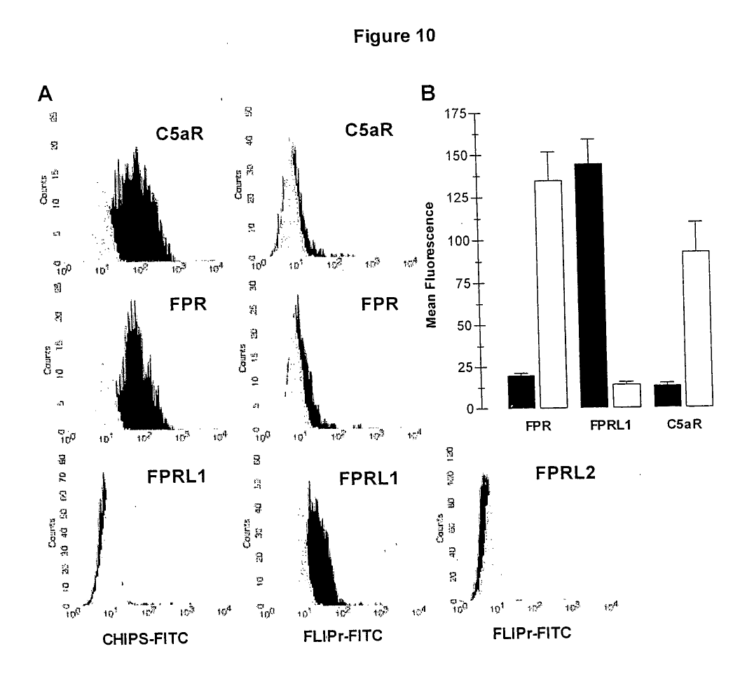

FIG. 10. FLIPr binds to HEK293 cells transfected with the FPRL1. HEK293 cells were transiently transfected with the vector containing FLAG-tagged human FPR, FPRL1 and C5aR or 3xHA-tagged FPRL2. As control, an empty vector was used. To identify positive transfectants, cells were labeled with anti-FLAG mAb (or anti-HA mAb for FPRL2) and APC-labeled goat anti-mouse IgG antibody. Simultaneously, FITC-labeled FLIPr or CHIPS was added at 3 μg/ml. Cells were resuspended in buffer with propidium iodide and analyzed for binding of FITC-labeled protein to viable, receptor-positive transfectants. Therefore cells were gated on basis of scatters and viability (propidium iodide negative) and analyzed for expression of the receptor on the cell surface (APC-positive) and binding of FITC-labeled protein. Figure A shows representative histograms of the binding of CHIPS-FITC to C5aR, FPR, and FPRL1 (left column) and FLIPr-FITC to C5aR, FPR, FPRL1, and FPRL2 (right column). Background staining to vector control cells is depicted as gray overlays. Figure B shows the mean fluorescence±SEM of three independent experiments; black bars represent FLIPr-FITC and open bars CHIPS-FITC binding. Mean fluorescence value for binding to vector control HEK293 cells was 8.6±1.

FIG. 11. FLIPr-like binds to neutrophils, monocytes and a proportion of lymphocytes. Isolated PMN and PBMC were incubated with a range of concentrations of FLIPr-like-FITC (0.03 to 2.60 μg/ml) for 30 minutes on ice (A) or at 37° C. (B) under constant shaking. Cells were then washed and resuspended in RPMI-HSA and fluorescence was measured in a flow cytometer. Cells were identified based on scatter parameters and anti-CD14 staining; neutrophils (), monocytes (▪) and lymphocytes (▴) are displayed. Data are from a representative experiments.

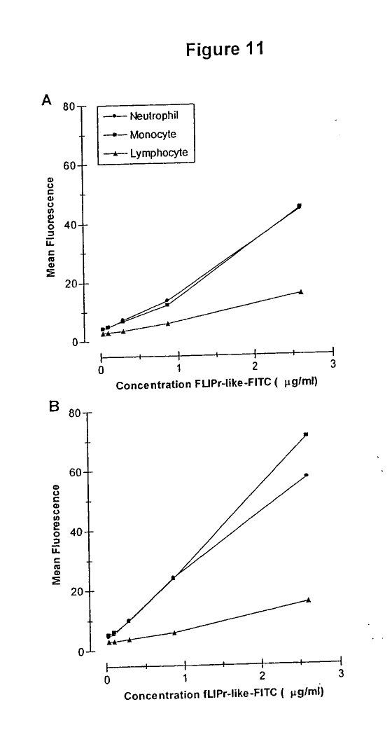

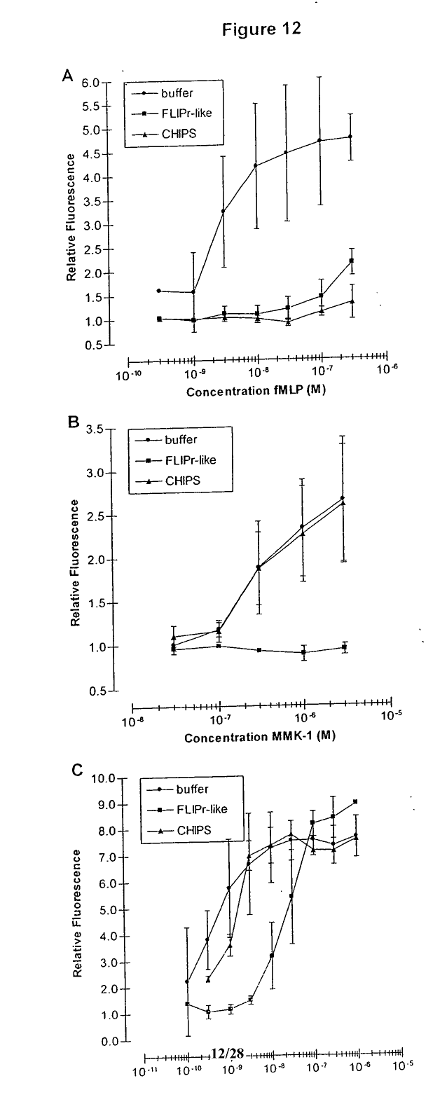

FIG. 12. FLIPr-like inhibits fMLP, MMK-1, and WKYMVm induced calcium mobilization in neutrophils. Fluo-3-loaded neutrophils were incubated with buffer (O), 3 μg/ml FLIPr-like (▪) or CHIPS (▴) for 20 minutes at room temperature. For calcium mobilization, each sample was first measured for about 10 seconds to determine the basal fluorescence and subsequently increasing concentrations fMLP (A), MMK-1 (B), or WKYMVm (C) were added and rapidly placed back in the sample holder to continue the measurement. Cells were analyzed in a flow cytometer and activation was expressed as the ratio of the fluorescence value before (cells acquired between T=5 till 7 seconds)/after addition of stimulus (cells acquired at T=12 till 14 seconds after stimulation). Data are mean±SEM of three independent experiments.

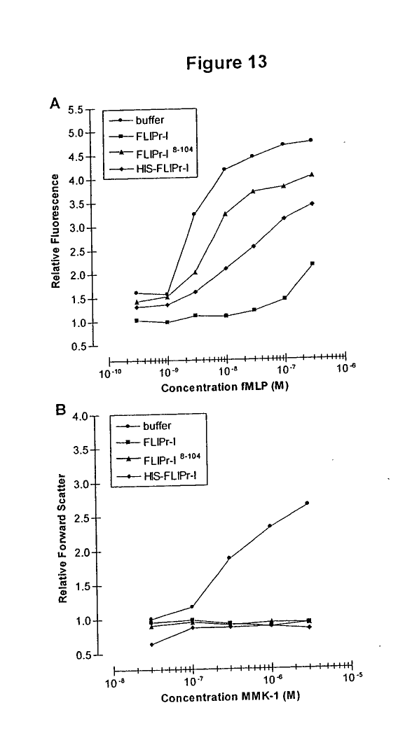

FIG. 13. Importance of the N-terminus of FLIPR-like in the fMLP- and MMK-1-induced calcium mobilization in neutrophils. Fluo-3-loaded neutrophils were incubated with buffer (), 3 μg/ml FLIPr-like (▪), deletion mutant FLIPr-like⁸⁻¹⁰⁴ (▴) or His-tagged FLIPr-like (♦). Cells were stimulated with increasing concentrations fMLP (A) or MMK-1 (B). Data are expressed as relative fluorescence from a representative experiment.

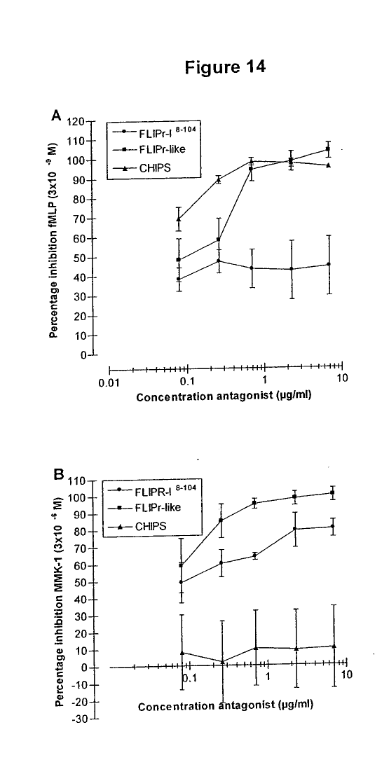

FIG. 14. Potency of FLIPr-like to inhibit the fMLP- and MMK-1-induced calcium mobilization in neutrophils. The activity of different concentrations CHIPS (▴), FLIPr-like (▪) and FLIPr-like⁸⁻¹⁰⁴ () was tested in calcium mobilization assays with neutrophils in response to synthetic peptide fMLP (3×10⁻⁹ M; A) and MMK-1 (3×10⁻⁶ M; B). Data are expressed as percentage inhibition and are the mean±SEM of three independent experiments.

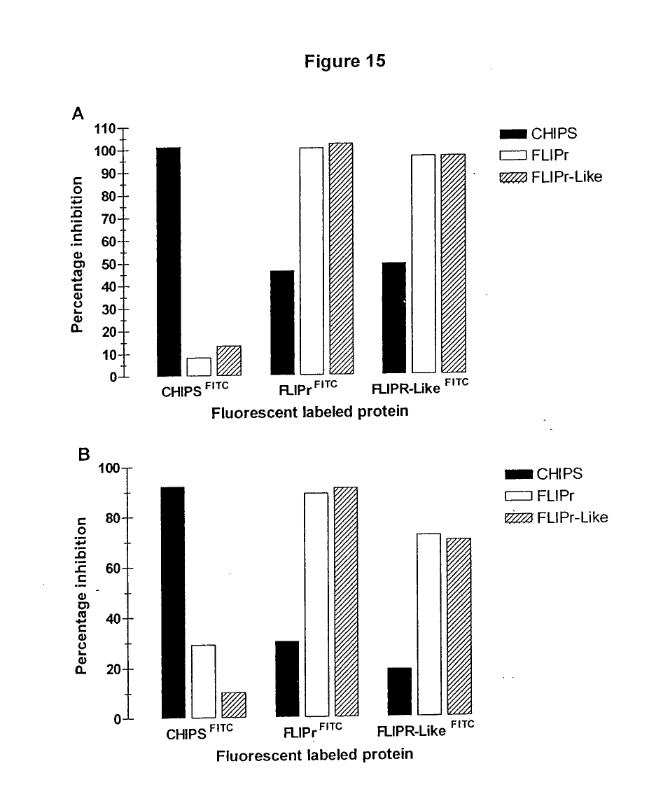

FIG. 15. FLIPr-like competes with FLIPr for binding to neutrophils and monocytes. The binding of fluorescent labeled antagonists (CHIPS, FLIPr and FLIPr-like) to neutrophils (A) and monocytes (B) was determined in the presence of unlabeled CHIPS (black bars, FLIPr (open bars) or FLIPr-like (hatched bars). Results are expressed as percentage inhibition and are the mean of four independent experiments. Inhibition is defined as 100 minus the MFL to cells with buffer—bgr MFL devided by MFL with competitor—bgr MFL.

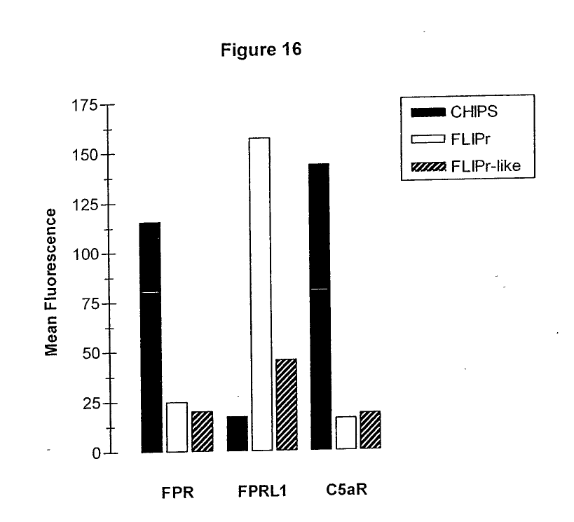

FIG. 16. FLIPr-like binds to HEK293 cells transfected with the FPR and FPRL1. HEK293 cells were transiently transfected with the vector containing FLAG-tagged human FPR, FPRL1 and C5aR. As control, an empty vector was used. To identify positive transfectants, cells were labeled with anti-FLAG mAb and APC-labeled goat anti-mouse IgG antibody. Simultaneously, FITC-labeled FLIPr-like, FLIPr, or CHIPS was added at 3 μg/ml. Cells were resuspended in buffer with propidium iodide and analyzed for binding of FITC-labeled protein to viable, receptor-positive transfectants. Therefore cells were gated on basis of scatters and viability (propidium iodide negative) and analyzed for expression of the receptor on the cell surface (APC-positive) and binding of FITC-labeled protein. Data are the mean fluorescence of a respresentative experiment; black bars represent CHIPS-FITC, open bars FLIPr-FITC, and hatched bars FLIPr-like-FITC binding. Mean fluorescence value for binding to vector control HEK293 cells was 8.6±1.

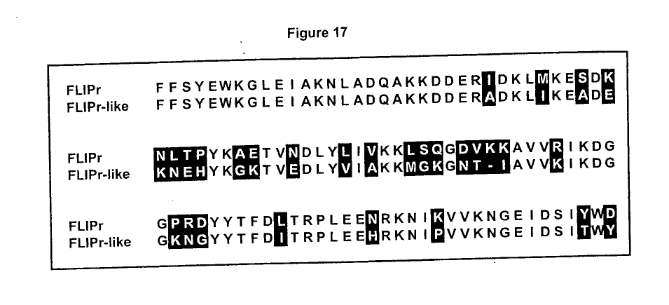

FIG. 17. Sequence alignment showing similarities between FLIPr and FLIPr-Like protein sequences. Sequences were aligned using clustal W. The shaded boxes mark mismatched residues. The first 25 amino acids of FLIPr and FLIPr-Like are similar. Most of the mismatched residues are located in the central part of the protein sequences.

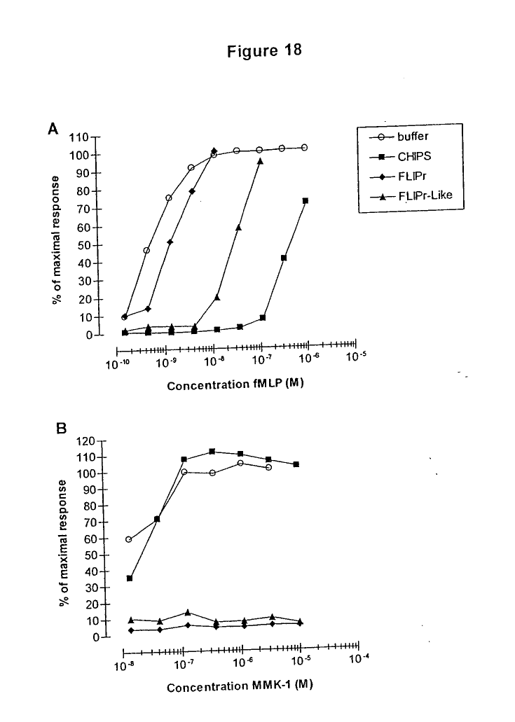

FIG. 18. FPR and FPRL-1 blocking activity of FLIPr, FLIPr-Like and CHIPS. Fluo-3 labeled isolated neutrophils were incubated with buffer (O), 1 μg/ml FLIPr (♦), FLIPr-like (▴) or CHIPS (▪). FMLP (A) and MMK-1 (B) induced activation was measured in a flow cytometer

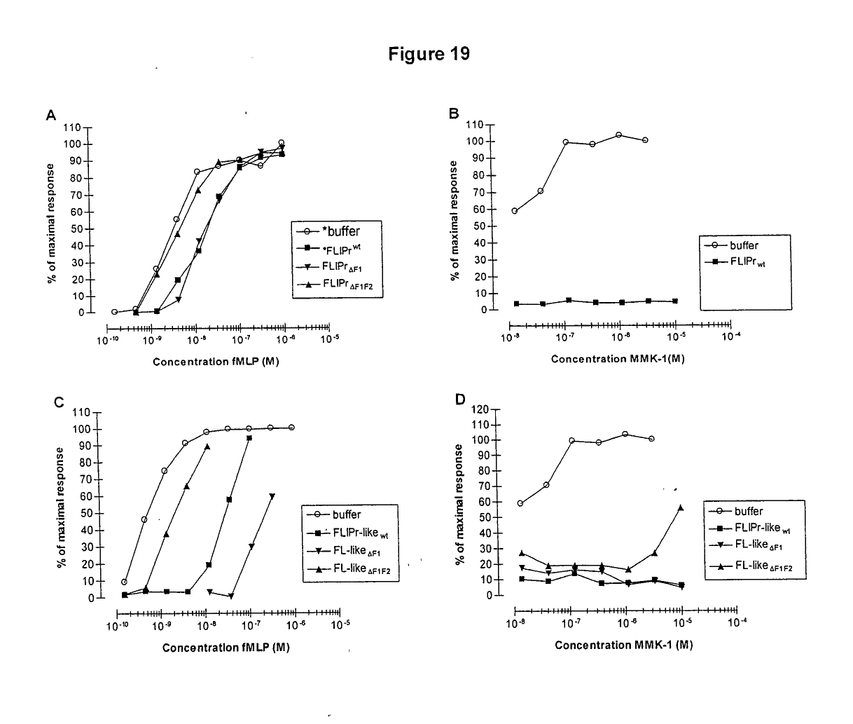

FIG. 19: FPR and FPRL-1 blocking activity of FLIPr-Like N-terminal mutants. The different recombinant proteins were tested in their ability to inhibit MMK-1 and fMLP induced activation of neutrophils. Fluo-3 labeled cells were incubated with 1 μg/ml of the sample protein and stimulated with different concentrations MMK-1 (A, C) or fMLP (B, D). Increase in fluorescence representing cell activation was measured in a flow cytometer.

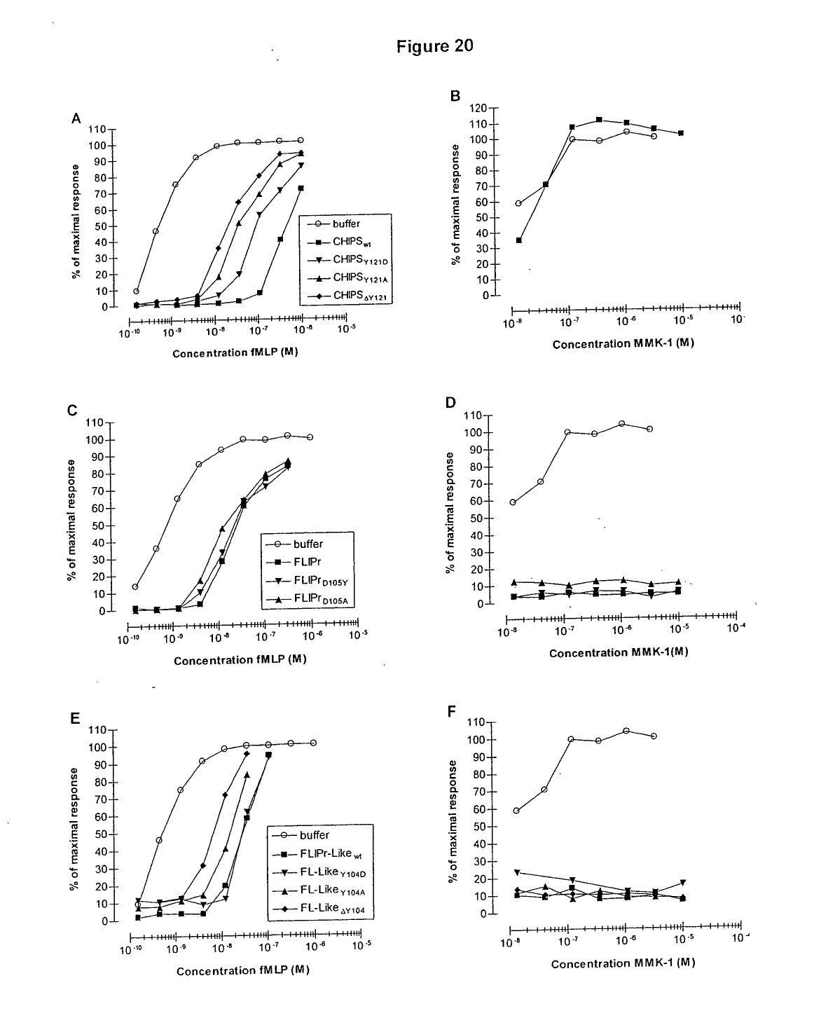

FIG. 20: FPR and FPRL-1 blocking activity of FLIP and FLIPr-Like C-terminal mutants. Different C-terminal substitution mutants of CHIPS, FLIPr and FLIPr-Like were tested for their ability to inhibit fMLP (A, C, E) or MMK-1 (B, D, F) induced activation of isolated neutrophils.

FIG. 21: FPR and FPRL-1 blocking activity of CHIPS and FLIPr-Like chimeras. Two different chimeras were created. FL-Like1-6-CHIPS a CHIPS protein in which the first 6 amino acids are substituted for the first 6 amino acids of FLIPr-Like and CH1-6-FL-Like the first six amino acids of FLIPR-Like substituted for CHIPS. The chimeras were tested in their ability to inhibit fMLP (A, C, E) or MMK-1 (B, D, F) induced activation of neutrophils.

The following abbreviations are used: Aβ, amyloid beta; CHIPS, Chemotaxis Inhibitory Protein of Staphylococcus aureus; C5aR, C5a Receptor; FPR, formyl peptide receptor; FPRL1, FPR-like receptor; GPCR, G protein-coupled receptor; LTB4, leukotriene B4; PAF, platelet activating factor; PrP, prion protein.

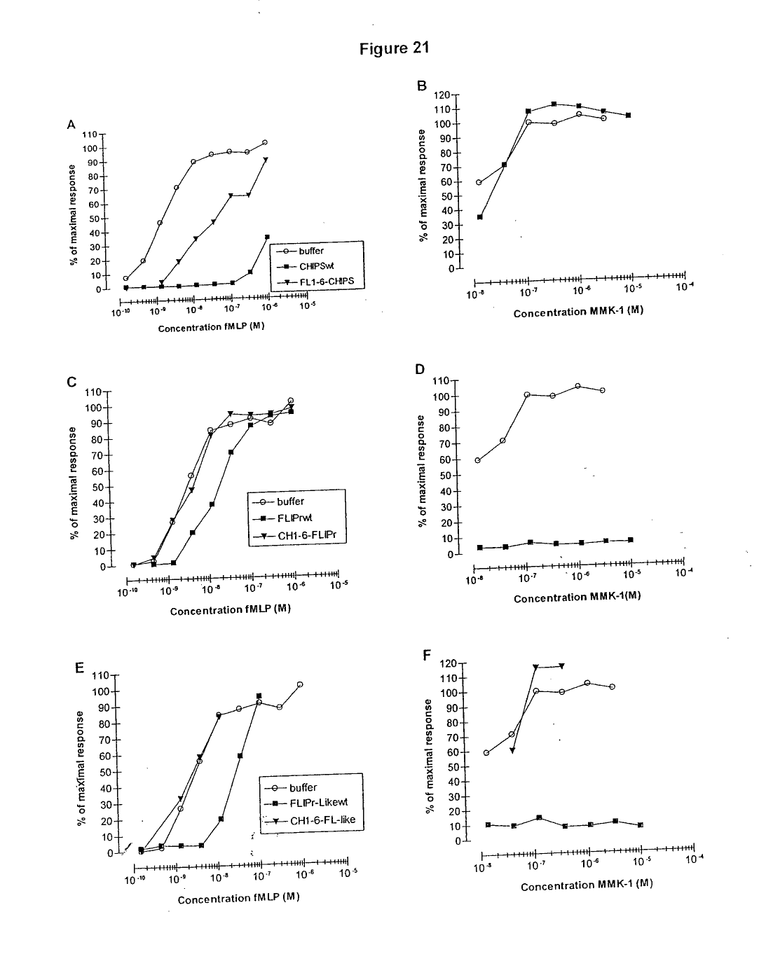

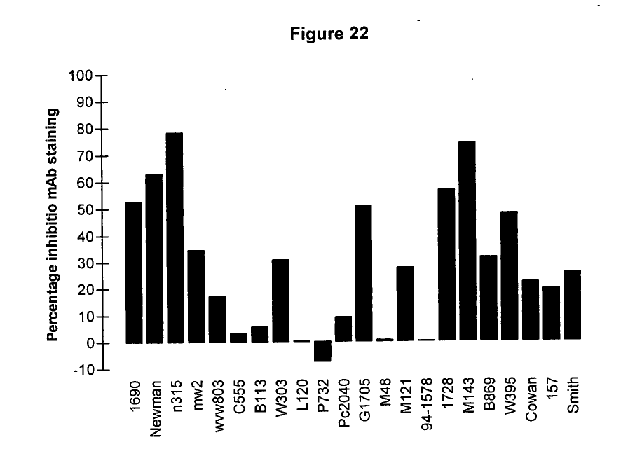

FIG. 22: Screening of Staphylococcal supernatants for inhibition of anti-CD32 staining on neutrophils. Human neutrophils were incubated with cell-free supernatants of S. aureus in a 1:1 (v/v) ratio. Subsequently, cells were stained with PE-labelled anti-CD32 mAb and analysed by flow cytometry. Results are expressed as percentage inhibition of the mean fluorescence value of buffer treated control cells.

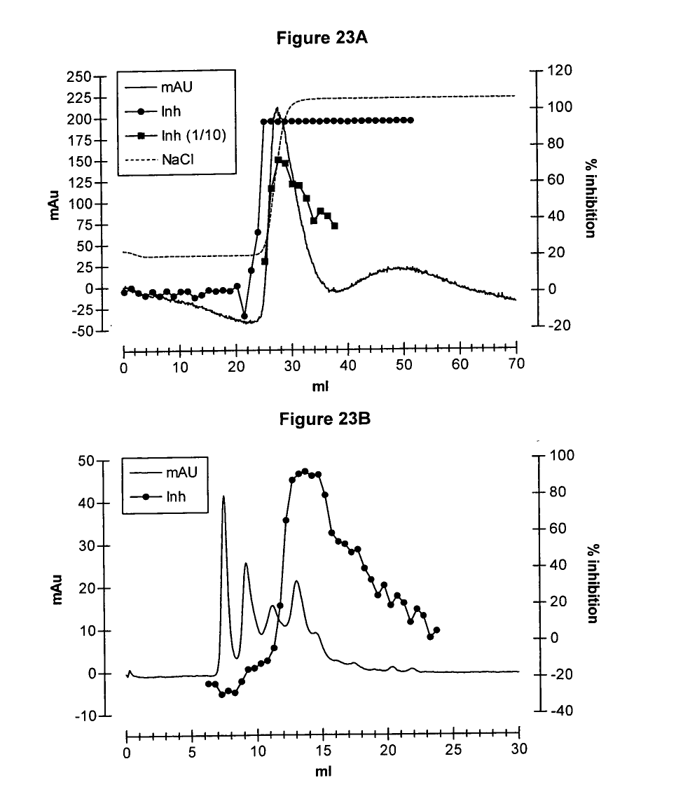

FIG. 23: Purification of anti-CD32 inhibitory activity in the supernatant of S. aureus.

A) A volume of 0.5 litre supernatant of the sequenced strain S. aureus subsp. aureus N315 was passed over a 25 ml Reactive-red ligand dye column and eluted with 1 M NaCl in fractions of 0.5 ml. Absorbance at 280 nm was recorded and fractions were screened for inhibition of anti-CD32 staining on neutrophils in a 1:1 () and 1:10 (v/v; ▪) dilution. The salt gradient of NaCl is indicated (--).

B) Pooled active fractions were concentrated, separated on a Superdex-75 column into 0.5 ml fractions and screened for activity in a 1:10 dilution ().

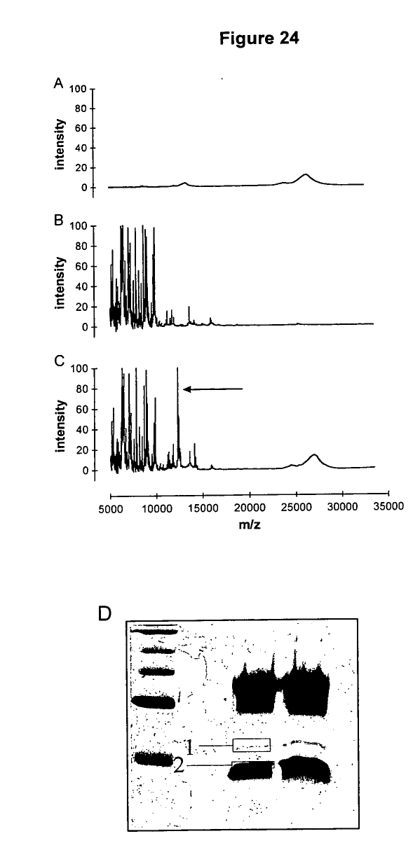

FIG. 24: Identification of anti-CD32 inhibitory activity by mass spectrometric analysis using SELDI-TOF and affinity isolation. Spectra from ProteinChip array coated with His-tagged CD32 and incubated with concentrated enriched S. aureus supernatant.

A) Spectrum from the array coated with CD32 and not incubated with the supernatant;

B) spectrum from the empty array incubated with the supernatant and

C) spectrum from the CD32-coated array incubated with the supernatant. The arrow points to the specific peak in molecular weight range of 5000 to 35000 Da. X-axis depicts m/z and y-axis the average intensity of ion peaks

D) Magnetic beads coated with His-tagged soluble human CD32 was used for selective capture of the CD32 inhibitory protein from the concentrated enriched S. aureus supernatant. Magnetic beads without CD32 were used as control. Beads were washed and bound material eluted into a small volume SDS-PAGE sample buffer. Proteins were run on a 15% SDS-PAGE and visualized by silver staining. Lane 1 contained molecular weight markers, lane 2 material from empty beads and lane 3 and 4 material from CD32-coated beads. The boxes 1 and 2 indicate the material that is excised for protein identification.

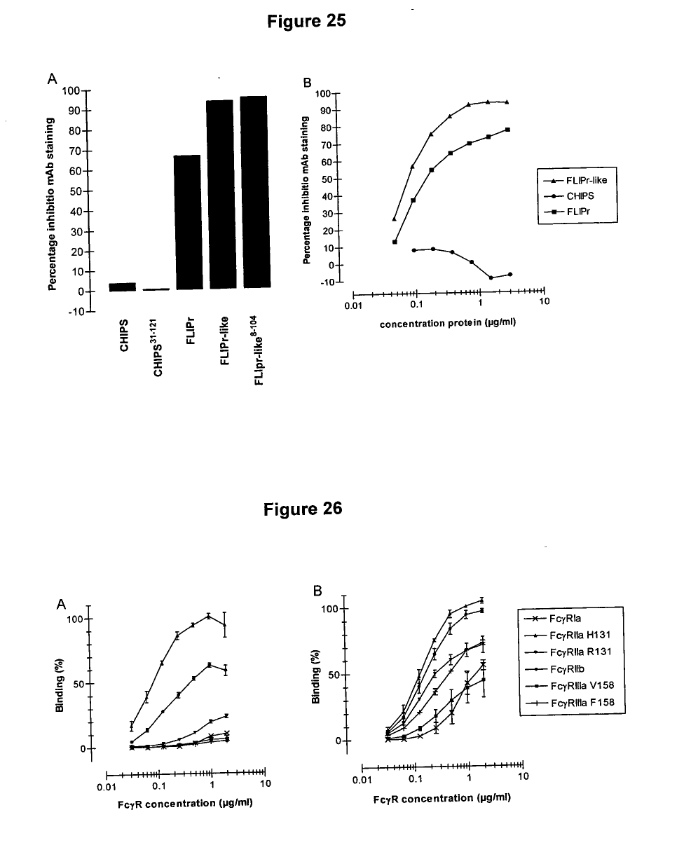

FIG. 25: Recombinant FLIPr and FLIPr-like inhibit anti-CD32 staining of neutrophils. Human neutrophils were incubated with FLIPr, FLIPr-like, FLIPr-like⁸⁻¹⁰⁴ mutant, CHIPS, CHIPS³¹⁻¹²¹ mutant or buffer control. Subsequently cells were stained with PE-labelled anti-CD32 mAb and analysed by flow cytometry. Results are expressed as percentage inhibition of the mean fluorescence value of buffer treated control cells. A) Individual proteins all at 1 μg/ml and B) concentration range.

FIG. 26: Binding of recombinant soluble Fcγ receptors to recombinant FLIPr and FLIPr-like by ELISA. FLIPr (A) and FLIPr-like (B) were coated to microtiterplates and incubated with a concentration range of the various His-tagged soluble FcγR. Bound FcγR was detected with a peroxidase labelled anti-HIS mAb and expressed relative to the signal obtained with 1 μg/ml high affinity FcγRIIa with Histidine at position131 (H131).

FIG. 27: Inhibition of ligand IgG binding to recombinant soluble FcγR by ELISA. His-tagged recombinant FcγR were captured with an anti-His mAb, incubated with different concentrations recombinant FLIPr, FLIPr-like, CHIPS or buffer control and analysed for binding of a fixed optimal concentration ligand IgG (HuMax-KLH). Results are expressed as percentage inhibition of control binding of HuMax-KLH to each individual FcγR; FcγRI (A), FcγRIIa H131 (B), FcγRIIa R131 (C), FcγRIIb (D), FcγRIIIa V158 (E), and FcγRIIIa F158 (F).

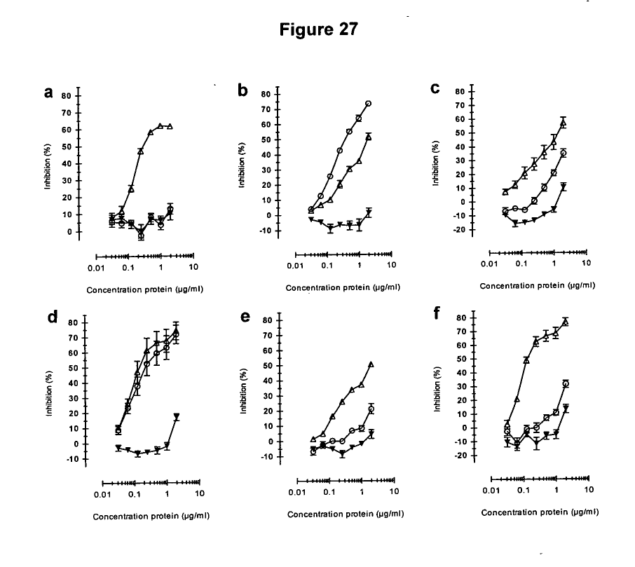

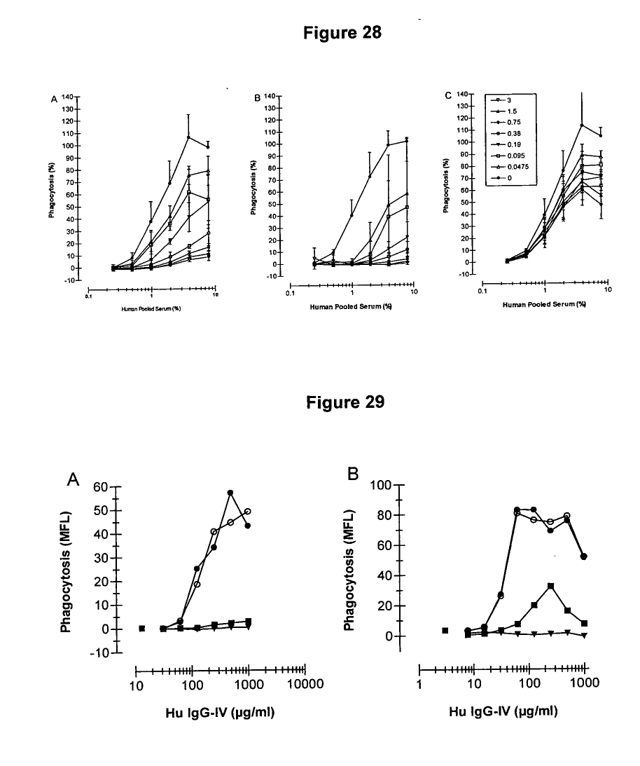

FIG. 28: Inhibition of IgG-mediated phagocytosis by human neutrophils. Neutrophils were incubated with different concentrations FLIPr (A), FLIPr-like (B), CHIPS(C) or buffer only for 15 min and subsequently mixed with fluorescent-labelled bacteria and a concentration range of heated human pooled serum as source for IgG. Phagocytosis was stopped after 15 min and neutrophil associated fluorescence measured by flow cytometry. Results are expressed as percentage of neutrophils that contain fluorescent-labelled bacteria (mean±SEM).

FIG. 29: Inhibition of IgG-mediated phagocytosis by human and mouse cells. Human neutrophils (A) and mouse macrophage P388D1 cell line (B) were incubated with FLIPr (▪), FLIPr-like (▴), CHIPS (◯) at 3 μg/ml or buffer () only and subsequently mixed with fluorescent-labelled bacteria and purified IgG for intravenous use. Phagocytosis was stopped after 15 min and neutrophil associated fluorescence measured by flow cytometry. Results are expressed as mean fluorescence values (MFL) of cells with bacteria minus background.

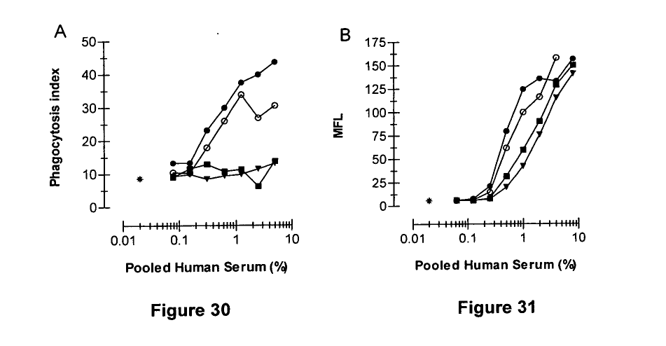

FIG. 30: Inhibition of phagocytosis by human monocytes. Human PBMC were incubated with FLIPr (▪), FLIPr-like (▴), CHIPS (◯) at 3 μg/ml or buffer () only and subsequently mixed with fluorescent-labelled bacteria and heated pooled human serum as IgG source. Phagocytosis was stopped after 15 min and cell associated fluorescence measured by flow cytometry using forward and sideward scatters to identify monocytes. Results are expressed as phagocytosis index defined as mean fluorescence values (MFL) of cells times percentage positive cells.

FIG. 31: Human neutrophil mediated phagocytosis with non-heated pooled human serum as source of both IgG and complement. Results are expressed as mean fluorescence of the cells (MFL).

### EXAMPLES

### Example 1

### Identification and Characterization of FLIPr

### Materials and Methods

### Reagents

MMK-1 (LESIFRSLLFRVM) was synthesized by Sigma-Genosys (Cambridge, UK). fMLP (N-formyl-methionyl-leucyl-phenylalanine), recombinant C5a, anti-FLAG mAb, propidium iodide and L-α-lysophosphatidyl-choline were from Sigma-Aldrich. WKYMVm was synthesized by Dr. John A W Kruijtzer (Department of Medicinal Chemistry, Utrecht Institute for Pharmaceutical Sciences, Utrecht, The Netherlands). WKYMVM, PrP106-126 and amyloid beta peptide Aβ1-42 were obtained from Bachem A G (Bubendorf, Switzerland). IL-8 and GRO-a were purchased from PeproTech (Rocky Hill, N.J.). Platelet activating factor (PAF-16) was from Calbiochem (La Jolla, Calif.). Leukotriene B4 (LTB4) was from Cayman Chemical (Ann Arbor, Mich.). Lipoxin A4 was from Biomol (Plymouth Meeting, Pa.). Fluo-3-AM (acetoxymethyl ester), Calcein-AM, Fura-red-AM, Fura-2-AM, and Alexa Fluo 488 Phalloidin were obtained from Molecular Probes (Leiden, Netherlands). Anti-HA mAb (clone 12CA5) was from Roche Applied Science (Penzberg, Germany).

Allophycocyanin (APC)-labeled goat anti-mouse Ig was from BD Pharmingen (San Jose, Calif.). Phycoerythrin (PE)-conjugated monoclonal antibodies CD4-PE (Leu-3a), CD8-PE (Leu-2a), CD19-PE (Leu-12), CD56-PE, CD16-PE and CD14-PE (Leu-M3) were obtained from Becton Dickinson (San Jose, Calif.); CD3-RPE-Cy5 (clone UCHT1) was from Dako (Glostrup, Denmark).

### DNA Sequence

The program tblastn with the nonredundant DNA database and the S. aureus genome database at http://www.ncbi.nlm.nih.gov was used to check for sequence similarities with the chp gene. A gene was found with a 49% homology with chp. The DNA sequence of the gene encoded a protein of 105 amino acids (in bold), preceded by a signal peptide and a signal-peptidase site (underlined):

MKKNITKTIIASTVIAAGLLTQTNDAKAFFSYEWKGLEIAKNLADQAKKD
DERIDKLMKESDKNLTPYKAETVNDLYLIVKKLSQGDVKKAVVRIKDGGP
RDYYTFDLTRPLEENRKNIKVVKNGEIDSIYWD

Primers were designed according to the published sequence of the gene (hypothetical protein SAV1156, Staphylococcus aureus subsp. aureus Mu50. GeneID: 1121132) for the cloning of the protein into pRSET vector (Invitrogen) and were manufactured by Invitrogen™ life technologies.
Prevalence in Clinical S. aureus Isolates

Prevalence of the gene for FLIPr (flr) was checked in 91 clinical and laboratory S. aureus isolates. Genomic DNA was isolated from cultures of S. aureus using the High pure PCR template preparation kit (Roche). PCR amplification was conducted using Supertaq polymerase (Enzyme Technologies Ltd, UK) and 5′-TTCTTTAGTTATGAATGGAA-3′ as the forward primer and 5′-TTAATCCCAATAAATCGAGTCG-3′ as the reverse primer. PCR products were detected by electrophoresis through agarose gel and ethidium bromide staining.

### Cloning and Expression of the Protein

The flr gene, without the signal sequence, was cloned into the pRSET vector directly downstream of the enterokinase cleavage site and in frame of the EcoRI restriction site by overlap extension PCR (Ho et al., Gene 77:51-59 (1989)). The plasmid pRSET was used as template for amplification of DNA fragments having overlapping ends using the sense primer 5′-GCTCTAGAAATAATTTTGTTTAACTTTAAGAAGGAG-3′ containing XbaI restriction site (underlined nucleotides) and the antisense primer 5′-TCTAAACCTTTCCATTCATAACTAAAGAACTTGTCGTCATCGTCGTACAG-3′. The gene was then amplified by PCR on chromosomal DNA of S. aureus Newman using the sense primer 5′-TTCTTTAGTTATGAATGGAA-3′ and the antisense primer 5′-CGTCCTGAATTCTTAATCCCAATAAATCGAGTCG-3′, containing the EcoRI restriction site (underlined nucleotides). The obtained DNA fragments were mixed, denatured and reannealed in a subsequent PCR reaction, using the primers corresponding to the 5′ and 3′ end sequences, in order to obtain the full-length PCR product.

The amplification reactions were performed using PfuTurbo DNA polymerase (Stratagene, Cedar Creek, Tex.). The final PCR product was purified using PCR Purification Kit (Qiaquick, Qiagen), cloned into the EcoRI and XbaI site of the pRSET vector and propagated in TOP10F′ E. coli following manufacturer's instructions (Invitrogen). After verification of the correct sequence by using ABI Prism 377 (Applied Biosystems), the recombinant protein was expressed in Rosetta-Gami E. coli (De3)pLysS (Novagen, MERCK Biosciences) by induction with 1 mM IPTG (Isopropyl β-D-thiogalactoside, Invitrogen).

### Purification and FITC-Labeling of the Protein

Bacteria were lysed with CelLytic B Bacterial Cell lysis/Extraction Reagent (Sigma) and lysozym according to the manufacturer's description. The histidine-tagged protein was purified using a nickel column (HiTrap™ Chelating HP, 5 ml, Amersham Biosciences) following the manufacturer's instructions and cleaved afterwards with enterokinase (Invitrogen).

Samples were checked for purity and presence of protein by means of 15% SDS-PAGE (Mini Protean® III System, Bio-Rad) and Coomassie Brilliant Blue (Merck) staining.

A portion of the protein was labeled with FITC (Sigma) for binding experiments. For that purpose, 500 mg/ml FLIPr was incubated with 50 mg/ml FITC in carbonate buffer pH 9.0 for 1 h at 37° C. under constant agitation. FLIPr-FITC was separated from unbound FITC using a desalting column (HiTrap™ desalting, Amersham Biosciences). The fractions were collected and tested for the presence of FLIPr (OD₂₈₀) and FITC(OD₄₉₅) in a spectrophotometer, to calculate the concentration: FLIPr-FITC (mg/ml)=[OD₂₈₀−(0.35×OD₄₉₅)]/1.547. Recombinant CHIPS was isolated, purified and FITC-labeled as described (de Haas et al., J. Exp. Med. 199:687-695 (2004)) using essentially the same procedures as for FLIPr.

### Leukocyte Isolation

Venous blood was collected from healthy volunteers into tubes containing sodium heparin. Blood was diluted with an equal volume of phosphate buffer saline (PBS) and layered onto a gradient of 12 ml Histopaque (density 1.117; Sigma Diagnostics) and 10 ml Ficoll (Amersham Biosciences) and centrifuged for 20 min at 379 g and 21° C. PBMC and PMN were collected separately from Ficoll and Histopaque interphases, respectively. Cells were then washed with cold RPMI-1640 (containing 25 mM Hepes and L-glutamine; Biowhittaker) with 0.05% human serum albumin (RPMI-HSA). For elimination of erythrocytes, the PMN pellet was subjected to a hypotonic shock by adding ice-cold H₂O for 30 seconds and subsequently adding ten-times concentrated PBS to reconstitute isotonicity, and washed afterwards. Cells were then resuspended to a concentration of 1.10⁷ cells/ml in RPMI-HSA.

### HEK293 Cells

Human embryonic kidney cells were transiently transfected with plasmids containing the DNA encoding a FLAG-tagged version of the human membrane receptors FPR, FPRL1 and C5aR or a 3XHA-tagged FPRL2. The DNA sequence of the receptors was amplified by PCR by using the following primer pairs:

for FPR sense primer
5′-CCGGAATTCATGGACTACAAGGACGACGACGACAAGATGATGGAGAC
AAATTCCTCTCTC-3′
and
antisense primer
5′-GCTCTAGATCACTTTGCCTGTAACGCCAC-3′;
for FPRL1 sense primer
5′-CCGGAATTCATGGACTACAAGGACGACGACGACAAGATGGAAACCAA
CTTCTCCACTCCTC-3′
and
antisense primer
5′-GCTCTAGATCACATTGCCTGTAACTCAG-3′;
for C5aR sense primer
5′-CCGGAATTCATGGACTACAAGGACGACGACGACAAGATGAACTCCTT
CAATTATACC-3′
and
antisense primer
5′-GCTCTAGACTACACTGCCTGGGTCTTCT-3′.

Primers contained EcoRI and XbaI restriction sites (underlined nucleotides). An N-terminal FLAG-tag (DYKDDDDK, included in the sense primers, bold nucleotides) was placed after the first methionine for detection by the anti-FLAG M2 mA.

The amplification reaction was performed on human bone marrow QUICK-Clone cDNA (BD Biosciences Clontech) using PfuTurbo DNA polymerase. The PCR product was digested with EcoRI and XbaI, ligated in the expressing plasmid pcDNA3.1 (Invitrogen) and transfected into HEK293 cells as described before (Postma et al., J. Biol. Chem. 280:2020-2027 (2005)).

The 3XHA-tagged FPRL2 DNA was obtained from UMR cDNA Resource Center (University of Missouri-Rolla, Rolla, Mo.) and was also transfected into HEK293 cells. HEK293 cells were grown in a 6-well plate (Costar, Corning, N.Y.) at 0.5×10⁵ cells/ml and maintained in EMEM (Minimal Essential Medium Eagle, BioWhittaker) supplemented with 0.1 mM nonessential amino acids, 1 mM sodium pyruvate, 10 mg/ml gentamycin and 10% fetal calf serum. After 3-4 days culture, cells were transfected with the respective plasmids by using Lipofectamine™ 2000 (Invitrogen), according to manufacturer's instructions. After two to three days from transfection, cells were used for binding assays.

### Calcium Mobilization

The activation of neutrophils by chemoattractants initiates a rapid and transient increase in the free intracellular calcium concentration. Calcium mobilization with isolated human neutrophils and monocytes was measured as previously described. In brief, the PMN fraction (5×10⁶ cells/ml) was loaded with 2 μM Fluo-3-AM or Fura-red-AM for 20 min at room temperature, protected from light and under constant shaking. The cells were then washed and resuspended in RPMI-HSA. Equal parts of cell suspension were incubated with buffer or protein (FLIPr, FLIPr-like, CHIPS, mutants or chimera) for 20 min. The cells (1×10⁶ cells/ml) were then monitored for calcium mobilization over time, first for 10 seconds to determine the basal fluorescence level, and then for 40 s after addition of the concentrated stimulus. Fluorescence was measured at 530 nm (for Fluo-3-AM) or 560 nm (for Fura-red-AM) using a flow cytometer (FACSCalibur or FACScan, Becton Dickinson). For calcium mobilization in PBMC, a PE-conjugated anti-CD14 was included during labeling with Fluo-2-AM. PBMC were adjusted to 5×10⁶ cells/ml and monocyte calcium mobilization was monitored by gating on side scatter and anti-CD14 staining. Results are expressed as relative fluorescence dividing the mean fluorescence of the peak fluorescence after stimulation by the basal mean fluorescence before challenge. Alternatively, data are expressed as a percentage of the maximal stimulation induced by the optimal stimulus concentration.

For ratiometry, neutrophils were labeled with Fura-2-AM for 45 min at room temperature, washed and resuspended with HBSS (BioWhittaker) containing 1% HSA at 7.5×10⁶ cells/ml. Cells were transferred into black clear bottom microtiterplates (50 μl) and preincubated for 5 min with 25 μl of inhibitory protein or HBSS—HSA buffer control and subsequently loaded into a FlexStation fluorescent plate reader (Molecular Devices). Fluorescence was measured every 1.5 seconds at dual wavelengths of 340 excitation with 530 and 590 emission. Stimuli were automatically added after a 1 min baseline reading and continued for an additional 5 min. The ratio of 530 to 590 was calculated for every reading and plotted versus time.

### Changes in Forward Scatter

Activation of neutrophils by fMLP results in a shape change that can be measured as change in forward scatter in a flow cytometer (Keller et al., J. Leukoc. Biol. 58:519-525 (1995)). Neutrophils (90 μl of a 2×10⁶ c/ml suspension) were incubated for 10 min at 37° C. in a shaking water bath together with 10 μl RPMI-HSA or inhibitory protein (FLIPr or CHIPS). Subsequently, different concentrations of ten-times concentrated stimulus were added, and the cells were incubated for another 15 min at 37° C. The cells were finally fixed with an equal volume of 2.5% glutaraldehyde (Merck) in saline, and kept on ice for at least 90 minutes before measurement in a flow cytometer. After appropriate gating to exclude cell debris, the forward scatter values were determined.

### Chemotaxis Assays

Chemotaxis of human neutrophils towards several chemoattractants was measured in a 96-multiwell trans membrane system (ChemoTX, Neuro Probe, Gaithersburg, Md.) with an 8 μm polycarbonate membrane. For labeling, neutrophils (5×10⁶/ml) were incubated with 2 mM Calcein-AM for 20 minutes at room temperature protected from light. Subsequently, cells were washed with HBSS containing 1% HSA (10 min, 1200 rpm), resuspended to 2.5×10⁶ cells/ml in the same buffer, and incubated with FLIPr. Dilutions of the different chemoattractants were prepared in HBSS—HSA, and 29 ml were placed into each well of the lower compartment of the chamber in triplicate.

Wells with control medium were included to measure the spontaneous cell migration and for total counts wells were filled with 25 ml of labeled cells plus 4 ml buffer. The membrane holder with 8 μm pore size was assembled, and 25 ml of labeled cells were added as a droplet to each upper well except for the total counts wells. The plate was incubated for 30 min at 37° C.+5% CO₂. The membrane was washed extensively with PBS and fluorescence of the wells was measured in a FlexStation Multiwell Fluorometer (Molecular Devices) with excitation at 485 nm and emission at 530 nm. Percentage of chemotaxis was calculated relative to the fluorescence value of cells added directly to the lower well: (fluorescence sample/fluorescence total counts)*100.

### Actin Polymerization

In order to measure the polymerization state of actin in neutrophils after proper stimulation, a flow cytometric assay was performed using fluorescent phallocidin as probe, which binds specifically to F-actin, the active state of actin. A set of tubes was prepared with 25 ml of fixation/permeabilization buffer (6% formaldehyde in PBS with 200 mg/ml L-a-lysophosphatidylcholine). Neutrophils (5×10⁶ cells/ml) with or without inhibitor were stimulated at room temperature with LTB4. The first sample (25 ml) was immediately added to a tube with fixation buffer, and consecutive samples at different time points. After keeping the samples for at least 15 min for fixation and permeabilization, 2 ml of the fluorescent probe (Alexa Fluo 488 Phallocidin, 100 U/ml in methanol) was added. Samples were then kept at 4° C. for 1 h and subsequently the fluorescence was measured on a flow cytometer.

Binding Assay with Leukocytes

To determine the binding of FLIPr to different cell types, isolated fractions of PMN and PBMC suspension were mixed again (4:6 ratio) and diluted to 5×10⁶ cells/ml with RPMI-HSA 1%. The cells were incubated with buffer or a concentration range of FITC-labeled protein during 30 min. Cells were then washed and resuspended in RPMI-HSA and binding of FLIPr was measured by flow cytometry. For binding in whole blood, 50 μl of EDTA anti-coagulated blood was incubated with 5 μl of different concentrations of FITC-labeled protein for 30 min at 4° C. Subsequently, samples were treated with FACS™ Lysing solution, washed once, and the cells were resuspended in 200 mL RPMI-HSA and measured in the flow cytometer. The same protocol was also used for isolated PBMC adding the appropriate monoclonal antibodies against different subsets of leukocytes, labeled with fluorochromes distinct from FITC: CD3-Cy5 plus CD4-PE or CD8-PE for T lymphocytes; CD19-PE for B-lymphocytes; CD14-PE for monocytes; CD3-Cy5 plus CD56-PE and CD16-PE for natural killer cells.

Binding Assay with HEK293

Cells transfected with each FLAG-tagged C5aR, FPR and FPRL1 or 3xHA-tagged FPRL2 were incubated with mouse anti-FLAG or anti-HA mAb (10 μg/ml) for 45 min at 4° C. Cells were then washed and incubated with APC-labeled goat anti-mouse antibody together with FITC-labeled FLIPr or CHIPS for 45 min at 4° C. Finally the cells were washed and resuspended in 200 μl of RPMI-HSA containing 5 μg/ml propidium iodide. Association of FITC-protein (FL1) was determined to propidium iodide negative living cells (scatters plus FL2) expressing the APC-positive tagged receptor (FL4) in a flow cytometer (26). For background signals, cells transfected with an empty pcDNA3.1 vector were used.

### Results

Prevalence in S. aureus Isolates

In order to investigate the prevalence of the gene for FLIPr (designated flr) in clinical isolates, 91 S. aureus strains isolated from bloodstream infections were screened by PCR. The gene encoding for FLIPr was found in 59% of the isolates.

FLIPr inhibits fMLP-induced activation of neutrophils The capacity of FLIPr to inhibit cell responses to chemoattractants was examined first. Incubation of human neutrophils with FLIPr resulted in the inhibition of fMLP-induced calcium mobilization (FIG. 1A) as well as changes in forward scatter (FIG. 1B). FLIPr itself, used as stimulus, did not induce a calcium response. Compared to CHIPS, it was found that the inhibition of fMLP-induced responses was weaker. The maximum inhibition of neutrophil activation was observed at the concentration of 3.10⁻⁹ M fMLP, while CHIPS inhibits up to 10⁻⁶ M fMLP. Unlike CHIPS, FLIPr did not block C5a-induced activation of neutrophils. In addition, FLIPr did not affect the response to other chemoattractant receptors present on neutrophils: LTB4, PAF, IL-8, and GRO-a (data not shown).

### FLIPr Inhibits Synthetic FPRL1 Agonist-Induced Activation of Neutrophils

Because FLIPr inhibited the fMLP-induced activation of neutrophils, its activity was also tested on the low-affinity receptor FPRL1. Several synthetic peptides derived from a random peptide library, which have been reported as agonists of FPRL1 (Hu et al. J. Leukoc. Biol. 70:155-161 (2001), Christophe et al., Scand. J. Immunol. 56:470-476 (2002), Bae et al., J. Leukoc. Biol. 66:915-922 (1999)) were tested as chemoattractants. Neutrophils were tested for activation with and without preincubation with 3 μg/ml FLIPr or CHIPS. A very strong inhibition of the FPRL1-specific MMK-1 peptide-induced activation of FLIPr-treated neutrophils was observed (FIG. 2A). FLIPr also inhibited WKYMVM- (FPRL1 and FPRL2 agonist) and WKYMVm- (FPR and FPRL1 agonist) induced responses in neutrophils (FIGS. 2B and 2C). The inhibition was stronger for WKYMVM.

While FLIPr inhibits the response to concentrations of 10⁻⁸M WKYMVm, it is able to inhibit up to 3×10⁻⁷ M when using WKYMVM. CHIPS did not show any activity in inhibiting the response to FPRL1 agonists.

FLIPr Inhibits Synthetic FPRL1 Agonist-Induced Activation of Monocytes

Monocytes also bear the receptors of the FPR-family including the FPR, FPRL1 and FPRL2 that is not present on neutrophils. The same set of agonists was used to stimulate the monocyte intracellular calcium mobilization in the presence of FLIPr or CHIPS. Specific monocyte response in the PBMC preparation was established by gating on side scatter and anti-CD14 staining. FIG. 3 shows that FLIPr efficiently inhibited the response induced by MMK-1 (FIG. 3C, specific for FPRL-1), both WKYMVm (FIG. 3B), and WKYMVM (FIG. 3D). CHIPS did not affect these responses. The fMLP-induced response of control monocytes showed a smaller window as compared to the response induced in neutrophils (FIG. 3A). Only CHIPS and not FLIPr inhibited the fMLP-induced calcium mobilization in monocytes.

### Potency of FLIPr

The FITC-labeled FLIPr was also functional in calcium mobilization assay (using Fura-red instead of Fluo-3-AM) inhibiting fMLP-, WKYMVm- and MMK-1-induced activation of neutrophils.

To further investigate the potency of FLIPr, an experiment was performed with a dose response of both FLIPr and MMK-1. The effect was dose-dependent and FLIPr inhibited the response to MMK-1 in the nanomolar to micromolar range (FIG. 4).

### FLIPr Inhibits Chemotaxis to FPRL1 Agonists

In order to assess if FLIPr could also inhibit the chemotactic response, the neutrophil migration in response to the chemoattractants C5a, fMLP, and MMK-1 was determined in a microwell chemotaxis assays. In accordance with the calcium mobilization assays, FLIPr did not show any effect on C5a. However, FLIPr partly inhibited the chemotactic response to fMLP and showed a complete inhibitory activity towards MMK-1 (FIG. 5).

### FLIPr Inhibits Aβ1-42- and PrP106-126-Induced Activation of Neutrophils

Neurodegenerative diseases are a group of central nervous system disorders characterized by neuronal dysfunction and accumulation of fibrillar material. The activation of monocyte-derived cells is thought to play a key role in the inflammatory process leading to the pathogenesis of many neurodegenerative diseases. Although the potential involvement of other cell surface receptors should not be excluded, FPRL1 has been proposed to mediate the migration and activation of monocytes and microglia induced both by Aβ1-42 15 and by a 20-amino acid fragment of the human prion protein PrP106-126 (Le et al.; J. Immunol. 166:1448-1451 (2001)).

The capacity of FLIPr to inhibit the responses to these ligands was examined. FLIPr inhibited the calcium mobilization in response to 10 μM Aβ1-42 and 50 mM of PrP106-126 (FIG. 6A). For comparison, the potent inhibition of MMK-1- and fMLP-induced calcium mobilization by FLIPr was performed in parallel. With Aβ1-42 a specific migration was induced that was partly inhibited by FLIPr (FIG. 6B). Because the Aβ1-42-induced calcium response as determined by Fluo-3 and flow cytometry were relatively weak, the experiment was repeated with Fura-2 labeled cells and ratiometry in a fluorescent plate reader (FlexStation). This enabled a more clear view on the Aβ1-42-induced calcium response that was completely inhibited by FLIPr (FIG. 6C). To demonstrate specificity of the response, the same cells were rechallenged after 5 min with PAF. This elicited a calcium mobilization in all cells, both treated with buffer and FLIPr.

FLIPr does not Interfere with Lipoxin A4 Activity on LTB4

Lipoxin A4 is an endogenous lipid-derived mediator generated at sites of inflammation that has been reported to bind FPRL1/LXA4R with high affinity. Unlike peptide chemotactic agonists, lipoxin A4 induces an anti-inflammatory signalling cascade that inhibits neutrophils migration and suppresses calcium mobilization upon challenge with other agonists. Lipoxin A4 was also tested as a direct FPRL1-agonist in the calcium mobilization assay. However, we were unable to elicit a calcium response in neutrophils or monocytes in response to fresh lipoxin A4; neither when assayed with Fluo-3 and flow cytometry nor with Fura-2 and ratiometry in a fluorescent plate reader.

To investigate a possible antagonistic effect of FLIPr for lipoxin A4, inhibition of LTB4-induced actin polymerization was measured. Cells incubated with 10⁻⁶ M lipoxin A4 showed a decreased actin polymerization in response to LTB4. Pre-incubation with FLIPr at different concentrations could not revert this effect. FLIPr itself did not inhibit the actin polymerization in response to LTB4, in accordance with the results obtained with calcium mobilization (FIG. 7).

### FLIPr Binds to Human Neutrophils, Monocytes and a Subpopulation of Lymphocytes

To show association of FLIPr with the appropriate blood leukocytes that bear FPRL1, fluorescent-labeled FLIPr was used. With neutrophils and monocytes a strong association of FLIPr-FITC was observed, while lymphocytes showed a weak binding (FIG. 8). With increasing concentrations of FLIPr-FITC, an increase in binding was observed, both when cells were incubated at 37° C. (FIG. 8A) and on ice (FIG. 8B). To test if binding was influenced by plasma component, the experiment was also performed using whole blood ex vivo. The results were not different from binding to isolated leukocytes (data not shown).

Monoclonal antibodies against different PBMC subtypes were used together with FLIPr-FITC to determine the binding profile of FLIPr to different cell populations (FIG. 9). Binding was observed to monocytes (CD14+, gated on scatters), B-cells (CD19+ lymphocytes), a subpopulation of CD8+ lymphocytes and natural killer cells (CD3−/CD56+/CD16+ lymphocytes). The CD8+ subpopulation that bound FLIPr was identified as natural killer cells (CD56+, CD8+). No binding was found to T-cells (CD3+ lymphocytes), or the CD4+ subset and the majority of CD8+ subset.

FLIPr Binds to HEK293 Cells Transfected with FPRL1

To assess whether FLIPr binds directly to the human receptor FPR and/or FPRL1, HEK293 cells transiently transfected with FLAG-tagged FPR and FPRL1 were tested for FLIPr-FITC binding. As positive controls, CHIPS-FITC binding and C5aR-transfected HEK293 were included. Cells were analyzed by gating on forward and sideward scatters as well as viability (cells staining negative for propidium iodide) to exclude dead cells. Indirect APC-labeled mAb against the FLAG or 3XHA tag detected the population of transfectants expressing the respective receptors. FIG. 10A shows representative histograms of the binding of FLIPr-FITC and CHIPS-FITC to the transfectants. As expected, CHIPS-FITC (3 μg/ml) bound to HEK293 transfected with FPR as well as those transfected with C5aR and did not bind to cells transfected with FPRL1. FLIPr-FITC (3 μg/ml) bound very strongly to HEK293 transfected with FPRL1, did not bind to HEK293 transfected with C5aR or FPRL2 and showed a weak binding to cells transfected with FPR. Binding to vector-control transfectants gave a Mean Fluorescence of 8.6±1.1 (FIG. 10B).

### Discussion

Leukocyte migration to the site of inflammation is a key event in the innate immune response to invading microorganisms. We describe FLIPr as a secreted staphylococcal protein that exerts anti-inflammatory activity by inhibiting calcium mobilization and cell migration towards chemoattractants. The experiments performed conclusively indicate that FLIPr uses FPRL1 as a functional receptor. FLIPr binds directly to HEK293 cells transfected with FPRL1. While fMLP is a high-affinity agonist for FPR, it interacts with and induces calcium mobilization through FPRL1 only at high concentrations. The slight binding of FLIPr-FITC to FPR requires further analysis, although FPRL1 possesses a 69% identity at the amino acid level with FPR). FLIPr inhibits very strongly the response to MMK-1, a potent and very specific FPRL1 agonist, but also to WKYMVM (FPRL1 and monocyte-expressed FPRL2 agonist). Finally, FLIPr inhibits the leukocyte responses to the reported host-derived FPRL1-agonists Aβ1-42 and PrP106-126.

The gene coding for FLIPr was found to be located in a genetic cluster which contains genes encoding several virulence factors: extracellular fibrinogen-binding protein (efb), extracellular fibrinogen-binding protein-like (efb-L), haemotoxin protein A (better known as a-toxine, hla), and enterotoxine-like proteins as well as an insertional sequence (tnp IS1181). Furthermore, the gene is present in 59% of clinical isolates.

The blocking of receptors for chemoattractants exerted by the staphylococcal proteins CHIPS and FLIPr may have a role in preventing the early detection of the microorganism by the innate immune mechanisms, allowing its spread.

Leukocyte migration is critical in maintaining the host defense, aiming at the clearance of noxious agents. Uncontrolled cellular infiltration into tissues can lead to chronic inflammation and toxic release of substances such as superoxide anions. FPRL1 constitutes an important molecular target for the development of new therapeutic agents to combat excessive inflammatory responses.

Furthermore, the activation of FPRL1 by Aβ1-42 or PrP106-126 leads to accumulation and activation of mononuclear phagocytes (monocytes and microglia) as well as fibrillar formation that is associated with the pathogenesis of Alzheimer's disease and prion diseases, respectively.

The Alzheimer patient will benefit from a combination of different drugs and the development of FPRL1-specific antagonists may have promising therapeutic potential in retarding the progression of the disease.

FLIPr is a novel bacterial evasion mechanism of S. aureus and a target for treatment of staphylococcal infections. Furthermore, as an FPRL1-specific antagonist, it provides new strategies for the development of anti-inflammatory agents in FPRL1-mediated diseases.

### Example 2

### Another Formyl Peptide Receptor Like-1 Inhibitor from Staphylococcus aureus (FLIPr-Like)

### Methods

### Reagents

The reagents are the same as used in Example 1.

### Cloning and Expression of FLIPr-Like

Primers were designed according to the published sequence of the gene for the cloning of FLIPr-like into pRSET vector (Invitrogen) and were manufactured by Invitrogen™ life technologies. A collection of clinical and laboratory S. aureus strains was screened for the presence of the gene by polymerase chain reaction (PCR) using the set of primers 5′-TTCTTTAGTTAT-3′ as sense primer and 5′-GCCGAATTCTTAATACCAAGTAATCGAA-3′ as reverse primer.

One of the positive strains was used as target DNA for cloning of the protein. Recombinant protein was generated by PCR and cloned into the EcoRI and XbaI site of the pRSET vector by overlap extension PCR as described above. Amplification was performed with Supertaq or Pfu DNA polymerase (Stratagene). The recombinant protein was propagated in TOP10 E. coli (Novagen). After verification of the correct sequence, the protein was expressed in Rosetta-Gami (DE3)pLysS E. coli (Novagen), by induction with 1 mM IPTG (Invitrogen). Expression of the protein was checked by SDS-PAGE (Mini Protean® 3 System, Bio-Rad) and Coomasie blue staining. Protein was present in the insoluble fraction and required the denaturating protocol for purification.

Bacteria were lysed with guanidine lysis buffer and urea was used for denaturating. The histidine-tagged protein was purified using a nickel column (HiTrap Chelating HP, 5 ml, Amersham biosciences) following manufacturer's instructions, and cleaved afterwards with enterokinase (Invitrogen), to separate the His-tag from the native protein. Initially the native protein was also bound to the column and could be eluted with EDTA buffer together with the His-tag. SDS PAGE of the samples with higher OD showed digested protein, so it was considered an unspecific binding to the column. The sample was dialyzed again into phosphate buffer, and flowed through the column the next day. Phosphate buffers with lower pH (pH 7.8, pH 6, pH 5.3) were successively flowed through and samples were collected every time.

A SDS-PAGE gel was run with the samples with the higher OD and two different bands of purified protein were observed, corresponding to 12 Kd and 11 Kd, respectively, and separated by means of the pH. The corresponding fractions were pooled and dialyzed separately towards PBS. The next day, OD was measured at 280 nm and concentration of the protein was calculated according to molar extinction coefficient. The two different protein fractions were blotted to paper, excised and sequenced at the Sequence Center Utrecht. The N-terminal sequencing identified the 12 Kd band as the native protein (FLIPr-like, first 5 N-terminal amino acids: FFSYE) and the 11 Kd band as a cleavage product without the first seven amino acids, FLIPr-like N-7 (underlined, first 5 N-terminal amino acids: GLEIA).

FFSYEWKGLEIAKNLADQAKKDDERADKLIKEADEKNEHYKGKTVEDLYV
IAKKMGKGNTIAVVKIKDGGKNGYYTFDITRPLEEHRKNIPVVKNGEIDS
ITWY

The native protein FLIPr-like was mixed with 0.1 mg/ml FITC (fluorescein isothiocyanate, Sigma) in 0.1M carbonate buffer pH 9.5 and subsequently separated from free FITC by a desalting column.

### Construction of FLIPr Mutants and Chimeras

Site-directed mutagenesis was performed on the FLIPr N-terminus by deletion of the first (FLIPr-DlF) or the first two (FLIPr-D1F2F) amino acids, both phenylalanines, and cloning in pRSET vector by overlap extension PCR as described above. Two chimeras were also constructed: CHIPS¹⁻⁶-FLIPr⁷⁻¹⁰⁴, in which amino acids 1-6 were substituted for amino acids 1-6 from CHIPS, and FLIPr¹⁻⁶-CHIPS⁷⁻¹²¹, in which amino acids 1-6 were from FLIPr and the rest of the molecule (7-121) was from CHIPS. The following 5′ primers were used to amplify, CHIPS¹⁻⁶-FLIPr⁷⁻¹⁰⁴, FLIPr¹⁻⁶-CHIPS⁷⁻¹²¹, FLIPr-D1F and FLIPr-D1F2F respectively: 5′-GTTTACTTTTGAACCGTTTAAAGGTTTAGAAATCGCAAA-3′, 5′-GTTCTTTAGTTATGAATGGCCTACAAATGAAGAAATAGA-3′, 5′-GTTTAGTTATGAATGGAAAGGTTTAG-3′ and 5′-GAGTTATGAATGGAAAGGTTTAG-3′. The following primers containing the EcoRI digestion site (underlined) were used as reverse primers: 5′-GTCCTGAATTCTTAATCCCAATAAATCGAGTCG-3′ for CHIPS¹⁻⁶-FLIPr⁷⁻¹⁰⁴, FLIPr-D1F and FLIPr-D1F2F, and 5′-GCTACTAGCTGAATTCTTAGTATGCATATTCATTAG-3′ for FLIPr¹⁻⁶-CHIPS⁷⁻¹²¹.

The competent cells BL21 (DE3) E. coli (Novagen) were used to express the mutants and chimeras. After verification of the correct sequence, all proteins were expressed and purified using a nickel column (ProBond Resin, Invitrogen) following manufacturer's instructions.

### Synthetic Peptides

Peptides with amino acids 1-6 from FLIPr and amino acids 1-6 from CHIPS were synthesized by Dr. R. van der Zee, Institute of Infectious Diseases and Immunology, Utrecht University, as described by Haas et al. (J. Immunol. 173:5704 (2004)).

### Leukocyte Isolation and Calcium Mobilization

The leukocyte isolation and calcium mobilization were performed as described in Example 1.

### HEK293 Cells

Human embryonic kidney cells were transfected with plasmids containing the DNA encoding a FLAG-tagged version of the membrane receptors FPR, FPRL1 and C5aR as described above.

### Binding Assays

To determine the binding of fluorescent-labeled proteins to different cell types, isolated fractions of PMN and MNC were mixed at a 4:6 ratio and diluted to 5×10⁶ cells/ml with 1 ml RPMI-HSA 1%. Subsequently, the cells were incubated with buffer or FITC-labeled protein in a range of concentrations during 30 min. Cells were then washed and resuspended in RPMI-HSA. The fluorescence of 17500 cells was measured by flow cytometry and the different leukocyte populations were identified based on forward and sideward scatter parameters. For binding in whole blood, 50 μl of EDTA anti-coagulated blood was incubated with 5 μl of different concentrations of FITC-labeled protein during 30 minutes at 4° C. Subsequently, samples were incubated with FACS™ Lysing solution and, after washing, pellet was resuspended in RPMI-HSA, and fluorescence measured in the flow cytometer.

### Binding Assays Using HEK293

This binding assay is the same as described in Example 1.

### Results

### FLIPr-Like Binds to Neutrophils, Monocytes and a Proportion of Lymphocytes

Neutrophils, monocytes and lymphocytes were gated based on forward and sideward scatters and the fluorescence intensity of FLIPr-like-FITC binding was quantified. Binding of FLIPr-like-FITC could be observed to neutrophils, monocytes and a proportion of lymphocytes in a similar way to that observed with FLIPr-FITC FIG. 11.

FLIPr-Like Inhibits fMLP-Induced Activation of Neutrophils More Potently than FLIPr

Incubation of neutrophils with FLIPr-like resulted in the inhibition of fMLP-induced calcium mobilization. The inhibition of the rise in [Ca²⁺] was dose-dependent, and lower concentrations of FLIPr-like were effective. Furthermore, while FLIPr inhibits 3×10⁻⁹M fMLP, FLIPr-like was able to inhibit up to 10⁻⁷M fMLP (FIG. 12A). Because the results mimicked the activity of CHIPS on fMLP, the ability of FLIPr-like to block the C5a-mediated calcium mobilization was also tested. FLIPr-like did not show any activity on C5aR, while CHIPS effectively inhibited (data not shown).

### FLIPr-Like Inhibits MMK-1 and WKYMVm Induced Activation of Neutrophils

We examined whether FLIPr-like could also block the activation of FPRL1 by specific ligands such as the synthetic peptides MMK-1 and WKYMVm. We tested the calcium mobilization in neutrophils, preincubated with FLIPr-like or CHIPS and compared that to control cells. FLIPr-like inhibited the cell response to MMK-1 and WKYMVm, while CHIPS was not effective (FIGS. 12B and C). Calcium mobilization assays were performed also with the FITC-labelled protein and using Fura-red-AM as a calcium probe, and its function was kept.

FLIPr-like⁸⁻¹⁰⁴ Inhibits MMK-1 Induced Responses and does not Inhibit fMLP-Induced Activation of Neutrophils

During the purification of recombinant FLIPr-like, a protein with 7 amino acids N-terminal deletion was generated and could be separately isolated from the intact protein. This enabled the possibility to investigate the importance of the N-terminus in FLIPr-like activity. The protein lacking the residues 1-7 (FLIPr-like⁸⁻¹⁰⁴) was also tested in their ability to block to fMLP and MMK-1-mediated calcium mobilization in neutrophils. While the MMK-1 blocking activity was completely intact, FLIPr-like⁸⁻¹⁰⁴ did not inhibit fMLP-induced activation. These results suggested a possible active site in the N-terminus for fMLP-mediated responses.

The same experiments were performed with the His-tagged version of the proteins (before enterokinase cleavage), both FLIPr and FLIPr-like, and both kept their activity on MMK-1 but lost it on fMLP, confirming the implication of the N-terminus (FIG. 13).

### Potency of FLIPR-Like

To further investigate the potency of FLIPr-like, an experiment was performed with neutrophils treated with increasing concentration FLIPr-like, FLIPr-like⁸⁻¹⁰⁴ and CHIPS stimulated with fMLP and MMK-1. The effect was dose-dependent and FLIPr-like as well as FLIPr-like⁸⁻¹⁰⁴ inhibited the response to MMK-1 in the nanomolar to micromolar range (FIG. 14). CHIPS only inhibited the fMLP response and not the MMK-1 response.

### Function of FLIPr Mutants and Chimeras

To further investigate which parts of the sequence are important in the activity of FLIPr, calcium mobilization assays were performed with several mutants, chimeras and peptides. The mutant of FLIPr lacking the first N-terminal amino acid showed similar activity as FLIPr with both fMLP and MMK-1, suggesting that the first phenylalanine is not important for its function.

The mutant lacking the first two N-terminal amino acids (both phenylalanines), lost its activity on both fMLP and MMK-1-induced responses (FIG. 19).

The peptide FLIPr 1-6, representing the first 6 amino acids of FLIPr, kept its activity on fMLP but lost the action on MMK-1 (FIG. 21). Because the first 6 amino acids of FLIPr closely resemble the allowed substitutions within the first 6 amino acids of CHIPS, chimeras were constructed that swap the initial 6 amino acids with the remaining sequence of FLIPr or CHIPS. Interestingly, the chimera CHIPS¹⁻⁶-FLIPr⁷⁻¹⁰⁴ had no activity on both fMLP and MMK-1, and the chimera FLIPr¹⁻⁶-CHIPS⁷⁻¹²¹ kept the activity on fMLP but lost it on MMK-1. These results are consistent with the hypothesis of the N-terminus as the active site of both FLIPr and FLIPr-like on fMLP-mediated responses, as shown for CHIPS. In addition, some part of the N-terminus seems to be important for the activity on MMK-1.

### FLIPr and FLIPr-Like Compete for the Same Binding Site

To compare the relative binding activities of both CHIPS, FLIPr and FLIPr-like for their respective receptors, binding of FITC-labeled proteins to neutrophils and monocytes was determined in the presence of unlabeled competitors. All three FITC-labeled proteins bound to both neutrophils and monocytes as shown before. Preincubation with the homologous unlabeled protein resulted in complete inhibition of the binding, both with neutrophils and monocytes. Furthermore, CHIPS preincubation partially inhibited binding of FITC-FLIPr and FLIPr-like to the cells, but not vice-versa. Unlabeled FLIPr and FLIPr-like were equally effective as competitor for the binding of FLIPr-FITC as well as FLIPr-like-FITC (FIG. 15).

FLIPr-Like Binds to HEK293 Cells Transfected with FPR and FPRL1

The FITC-labeled protein was used in binding experiments with HEK293 cells transfected with FLAG-tagged versions of FPR, FPRL1 and C5aR. The C5aR and an empty vector were used as controls. HEK293 cells were gated based on forward and sideward scatter parameters as well as viability, and only cells within these regions were analyzed for expression of the receptor.

Finally, the cells expressing the different receptors were analyzed for binding of the FITC-labelled proteins. FLIPr-FITC and CHIPS-FITC were used as controls. FLIPr-like-FITC bound to HEK293 transfected with FPRL1, and also FPR (FIG. 16).

### Discussion

The novel protein FLIPr-like presents a binding pattern and a function very similar to FLIPr. FLIPr-like shares with FLIPr the signal peptide and the first twenty-five amino acids. Furthermore, in the screened S. aureus isolates, the gene encoding FLIPr-like was present in strains that did not contain the gene encoding FLIPr.

The cleavage product of FLIPr-like lacking amino acids 1-7 conserved the blocking activity on MMK-1 mediated activation of neutrophils, but lost its activity on fMLP. This demonstrates that different active sites within the protein are responsible for inhibiting fMLP and MMK-1 induced responses, respectively. As confirmed with experiments with the peptides, mutants and constructs, the function of inhibition of fMLP-induced responses resides in the N-terminus.

### Example 3

### Common Aspects of Formyl Peptide Receptor Antagonists

In this example it is shown that the N-terminus of FLIPr-Like plays an important role in the activity towards both the FPR and FPRL-1. Aromatic amino acids in the N- and C-terminus of both CHIPS and FLIPr-Like are crucial for FPR blocking activity. Despite these similarities between CHIPS and FLIPr-Like experiments with CHIPS/FLIPr-Like, chimeras indicate that the two have different mechanisms of action. The sequence homology between the native FLIPr and FLIPr-like proteins is shown in FIG. 17.

### Materials and Methods

### Reagents

The same reagents were used as in Example 1.

Cloning, expression and purification of recombinant proteins Different recombinant proteins were cloned and expressed as described above. These proteins included:(i) CHIPS and CHIPS mutants with a substitution or deletion of the C-terminal amino acid (CHIPS^{Y121D}, CHIPS^{Y121A}A and CHIPS^{ΔY121}) (ii) FLIPr and FLIPr mutants with a substitution or deletion of the C-terminal amino acid (FLIPrD105Y and FLIPr^{D105A}) (iii) FLIPr-Like and FLIPr-Like mutants (FLLike^{Y104D}, FL-Like^{Y104A}, and FL-Like^{ΔY104}). The genes were cloned into the PRSET-B vector directly downstream the enterokinase cleavage site and before the EcoRI restriction site by overlap extension PCR.

Initially the FLIPr and FLIPr-Like genes were amplified from chromosomal S. aureus DNA. These products were used as template for further cloning. The amplification reactions were performed using Pfu Turbo DNA polymerase (Stratagene, Cedar Creek, Tex.). The final PCR product was purified using PCR Purification Kit (Qiaquick, Qiagen), cloned into the EcoRI and XbaI site of the pRSET-B vector and propagated in TOP10F′ Escherichia coli following the manufacturer's instructions (Invitrogen).

After verification of the correct sequence by using ABI Prism 377 (Applied Biosystems), the recombinant protein was expressed in Rosetta-Gami E. coli (Novagen, MERCK Biosciences) by induction with 1 mM IPTG (isopropyl β-D-thiogalactoside, Invitrogen). Bacteria were lysed with CelLytic B Bacterial Cell lysis/Extraction Reagent (Sigma) and lysozym according to the manufacturer's description. The histidine-tagged protein was purified using a nickel column (HiTrap Chelating HP, 5 mL, Amersham Biosciences) following the manufacturer's instructions and cleaved afterwards with enterokinase (Invitrogen). Samples were checked for purity and presence of protein by means of 15% SDS-PAGE (Polyacrylamide gel electrophoresis, Mini Protean R3 System, Bio-Rad) and Coomassie Brilliant Blue (Merck) staining. Protein concentrations were determined by absorbance at 280 nm.

### Isolation of Human Neutrophils and Calcium Mobilization

The same methods were followed as described in Example 1.

### Results

FLIPr-Like Inhibits MMK-1 and fMLF-Induced Activation of Neutrophils FLIPr and CHIPS are the two closest sequence homologues of FLIPr-Like. FLIPr inhibits MMK-1-induced neutrophil activation by blocking the FPRL-1. CHIPS binds the FPR and C5aR thereby inhibiting the fMLF- and C5a-induced activation of neutrophils. We tested the effect of FLIPr-Like on MMK-1, fMLF and C5a activation of neutrophils. FIG. 18 shows that FLIPR-Like inhibits the MMK-1 and fMLF induced activation. FLIPr-Like blocks both the FPR and FPRL-1 and thereby shares properties of both FLIPr and CHIPS. When we compare the activity of FLIPr-Like with FLIPr and CHIPS we see that FLIPr and FLIPr-Like have the same activity for blocking the FPRL-1. The FPR blocking activity of FLIPr-Like is approximately a 100-fold less compared to CHIPS.

### FLIPr-Like N-Terminus Plays a Role in Both FPR as FPRL-1 Blocking Activity

The phenylalanines at position 1 and 3 in CHIPS are crucial for FPR blocking activity. FLIPr and FLIPr-Like share a 100% sequence homology of the first 25 amino acids and both sequences start with two phenylalanines. In order to determine the role of the N-terminus in blocking the FPR and FPRL-1 we created FLIPr and FLIPr mutants with a deletion of the first or the first two phenylalanines. FLIPr-LikeΔF1 shows no decrease in FPRL-1 blocking activity (FIG. 19D). However, when we delete the first two phenylalanines (FLIP-likeΔF1F2 we see a decrease in FPRL-1 blocking activity. In contrast to this small decrease in activity, deletion of both phenylalanines completely abbrogates the FPR blocking activity of FLIPr-Like (FIG. 19D) and FLIPRr (FIG. 19A). Therefore, like in CHIPS, the N-terminus of FLIPr-Like plays an important role in activity.

C-terminus of Chips and FLIPr-Like Play a Role in FPR Blocking Activity

We showed that the N-terminal phenylalanines of CHIPS, FLIPr and FLIPr-Like are important for FPR and FPRL-1 blocking activity of these proteins. Earlier we reported that although the first 30 amino acids of CHIPS are poorly defined the N terminus is not completely disordered and might interact with the folded core of the protein. In this case the N-terminus CHIPS is in close proximity to the C-terminus. When we take a closer look at the C-termini of CHIPS, FLIPr and FLIPr-Like we see that both CHIPS and FLIPr-Like have a C-terminal tyrosine while FLIPr ends with an aspartic acid. Aromatic amino acids in both the N-terminus and the C-terminus may be involved in FPR blocking activity. To confirm this hypothesis we tested the activity of different C-terminal deletion and substitution mutants of CHIPS, FLIPr and FLIPr-Like on MMK-1 and fMLF induced activation of neutrophils (FIG. 20). CHIPSΔ121Y (CHIPS with a deletion of the C-terminal tyrosine) shows a decrease in FPR blocking activity compared to wild type CHIPS. Deletion of the C-terminal tyrosine in FLIPr-Like has a similar effect. FLIPr-LikeΔ104Y shows a decrease in FPR but not in FPRL-1 blocking activity.

This indicates that the C-termini of both CHIPS and FLIPr-Like are involved in FPR blocking activity. The presence of a folded core is essential for the FPR blocking activity demonstrated by a synthetic peptide comprising the N- and C-terminus of the CHIPS protein that showed no FPR blocking activity (data not shown). Although deletion of the C-terminal residue leads to a decrease in FPR blocking activity this is not always true when we substitute this amino acid. As shown in FIG. 20, substitution of Y104 in FLIPr for aspartic acid has no effect on activity. This is equally true for the C-terminal tyrosine in the CHIPS protein (FIG. 20A). The C-terminal residue of FLIPr-Like plays no role in FPRL-1 inhibitory activity (FIG. 20B). FLIPr-LikeΔ104Y has the same FPRL-1 blocking activity as wild type FLIPr-Like. This is also true for FLIPr because a deletion of D105 in FLIPr also has no effect on FPRL-1 blocking activity (FIG. 20C). Despite the high degree of sequence homology between the FLIPr and FLIPr-Like proteins substitution of the C-terminal aspartic acid in FLIPr with a tyrosine (as in FLIPr-Like) did not introduce FPR blocking activity.

### CHIPS/FLIPr-Like Chimeras

Although the activity of FLIPr-Like is less than CHIPS, both proteins show FPR blocking activity. Furthermore, we showed that in both proteins the N-terminal phenylalanine and, to a lesser degree, the C-terminal tyrosine play an important role in this activity. To further investigate the similarities between the CHIPS and the FLIPr-Like proteins we created two chimeras. In an earlier study we showed that a CHIPS derived peptide comprising the first 6 N-terminal amino acids (FTFEPF) was still able to block the FPR with a 10000 fold decrease in activity compared to wild type CHIPS. Therefore we substituted the first 6 amino acids of FLIPR-Like (FFWYEW) with those of CHIPS(CH¹⁻⁶-FL-Like) vice versa (FL-Like ¹⁻⁶-CHIPS) and tested these protein chimeras for FPR blocking activity as shown in FIG. 21.

CH¹⁻⁶-FL-Like completely lost the ability to inhibit both the FPR and FPRL-1 (FIGS. 21E,F). In contrast, FL-¹⁻⁶-CHIPS still possesses FPR blocking activity comparable to wild type FLIPr-Like activity (FIG. 21A). Also substitution of the N-terminus of FLIPr (CH¹⁻⁶-FLIPr) completely abrogates the FPR blocking activity (FIG. 21C).

### Discussion

FLIPr-Like, a protein excreted by S. aureus acts on both members of the formyl peptide receptor family (FPR and FPRL-1). The gene encoding FLIPr-like was found to be located on the same possible pathogenicity island as FLIPr together with other genes encoding virulence factors. Similar to CHIPS it was found that the N-terminal phenylalanines in FLIPr and FLIPr-like are crucial for their FPR and FPRL-1 blocking activities. Furthermore, the C-terminal tyrosine in CHIPS and FLIPr also play a role in FPR blocking activity. This shows that aromatic amino acids play an important role in the FPR blocking activity of both CHIPS and FLIPr-like. In both CHIPS and FLIPr the very first and very last amino acids are involved in function. Despite these similarities between CHIPS and FLIPr-like, experiments with CHIPS/FLIPr-like chimeras show CHIPS and FLIPr-like act by two different mechanisms. FLIPr-like in which the first 6 amino acids were substituted for CHIPS completely lost FPR blocking activity. In contrast, a CHIPS protein with the first 6 amino acids of FLIPr-like still showed FPR blocking activity. Although, here is some sequence homology between CHIPS and FLIPr-like large parts within the folded core of the CHIPS protein do not align with FLIPr-like. Together with CHIPS and FLIPr, FLIPr-like may provide an important immune evasion mechanism of S. aureus acting on the family of formyl peptide receptors. Although an inflammatory response is necessary clearing tissue debris and wound healing an exacerbated inflammatory response could cause further increase in tissue damage. Inhibition of phagocyte recruitment by inhibiting formyl peptide receptors could help to prevent this exaggerated inflammatory response.

### Example 4

### Inhibition of Fcgamma Receptor Function by FLIPr and FLIPr-Like

### Materials and Methods

### Initial Screening for Anti-CD32 Activity

Several strains of Staphylococcus aureus collected from patients (UMC-Utrecht and others) and laboratory strains were screened for possible activity. Therefore bacteria were cultured for 18 hours at 37° C. in Phenol Red negative IMDM containing L-Glutamine and 25 mM HEPES (Gibco, Invitrogen), centrifuged for 30 min at 4000 g, the supernatant collected and filtered over a 0.2 μm pore size filter to remove residual bacteria.

Part of the supernatant was dialysed in a 10 kDa membrane (Servapor; Serva) against PBS before storage at −20° C. (Veldkamp et al., Inflammation 21:541-551 (1997). Neutrophils were isolated from heparinized blood of healthy volunteers via a Histopaque-Ficoll gradient as described (Prat et al., J. Immunol. 177: 8017-8026 (2006)). The remaining erythrocytes in the neutrophil fraction were lysed for 30 seconds with sterile water and washed after reconstitution of the isotonicity. The cells are finally resuspended in ice-cold PRMI containing 25 mM HEPES (Gibco, Invitrogen) with 0.05% Human Serum Albumin (RPMI/HSA).

Cells (25 μl cells of 5×10⁶ cells/ml) were incubated with 25 μl Staphylococcal supernatant for 30 min on ice. Thereafter 5 μl PE-labeled anti-CD32 mAb 7.3 (RDI division of Fitzgerald Industries Intl, Concord Mass.) was added, incubated for another 30 min on ice and washed with RPMI/HSA. Samples were analysed for inhibition of anti-CD32 staining on a flow cytometer (FACScan or FACSCalibur; Becton Dickinson) and expressed as mean fluorescence value of 5000 neutrophils. Additional anti-CD32 mAb were also tested in combination with a FITC-labelled anti-mouse IgG: clone IV.3, 41H16, AT10 and 3E1.

### Enrichment of Anti-CD32 Activity

Staphylococcus aureus subsp. aureus N315 (a sequenced strain; GenBank BA000018) was cultured overnight in IMDM medium and the supernatant collected, filtered over a 0.2 μm filter and used immediately or stored at −20° C. A quantity of 1 liter of supernatant was passed over a 25 ml “Reactive Red 120” ligand dye cross-linked 4% beaded agarose column (Sigma-Aldrich) hooked onto an Akta-FPLC system (GE Healthcare Life Sciences). After washing with PBS the column was eluted with 1 M NaCl into fractions of 2.5 ml. PMSF (1 mM) was added and fractions were dialysed in PBS for 18 hours.

Fractions were screened for activity by anti-CD32 mAb staining on human neutrophils. Active fractions were pooled, concentrated with a 10 kDa Centriprep (Amicon, Millipore) and separated on a Pharmacia Superdex-75 gel filtration column into 2.5 ml fractions that were again screened for activity. Active fractions were pooled and concentrated using a 10 kDa Centriprep and stored at −20° C. in small aliquots.

Different preparations were precipitated with 20% TCA (trichloroacetic acid) for 30 min on ice and analysed on a 15% SDS-PAGE (Mini-Protean II; BioRad) by silver staining.

### Affinity Isolation of Anti-CD32 Activity

Magnetic Cobalt-chelating beads (TALON Dynabeads, Invitrogen) were coated with recombinant His-tagged human CD32a (the extracellular domain Ala 36-Ile 218 of human FcγRIIa; # 1330-CD, R&D Systems). Therefore 50 μl beads were washed twice with PBS containing 0.1% Triton-X100 (PBS-Triton) and incubated for 30 min with 100 μl of 200 μg/ml His-tagged CD32 in PBS. Beads were washed three times with PBS-Triton and incubated with purified supernatant for 18 hours at 4° C. under gentle rotation in a total volume of 400 μl. Supernatant was discarded and beads washed three times with PBS-Triton, suspended in 30 μl SDS-PAGE sample buffer for 15 min and heated for 2 min at 100° C.

The sample was briefly centrifuged (10 seconds at 10.000 g) and the supernatant analysed on a 15% SDS-PAGE by silver staining. Bands were excised and send for protein identification at the Department of Biomolecular Mass Spectrometry (Utrecht Institute for Pharmaceutical Sciences).

### Surface-Enhanced Laser Desorption Ionisation Time-of-Flight Mass Spectrometry (SELDI-TOF-MS)

For identification by mass, the Ciphergen (BioRad) IMAC30 ProteinChip Array was used that incorporates nitrilotriacetic acid groups forming stable complexes with metal ions. The array was loaded with 0.1 M nickel sulphate for 10 min under vigorous shaking, washed with de-ionised water, incubated with PBS for two times 5 min and incubated with 50 μl of 10 μg/ml His-tagged CD32 for 30 min under vigorous shaking.

The array was washes three times for 5 min with PBS, briefly rinsed with de-ionised water, air dried and treated with a saturated solution of SPA (sinapinic acid) as energy absorbing molecule that assists in desorption and ionisation.

Alternatively, the preactivated surface RS100 ProteinChip array was used to covalently immobilize CD32 (100 μg/ml) for 2 hours at room temp in a humidified chamber. The array was blocked for 1 hour with 0.5 M ethanolamine pH 8.5, washed with PBS and PBS containing 0.1% Triton-X100 under vigorous shaking.

Sample was incubated for 1 hour, washed with PBS/Triton-X100, rinsed with water and SPA added to each spot. After air-drying the array was analysed using the Ciphergen ProteinChip System Series 4000 read at a setting optimised for low molecular weight range. Spectra were externally calibrated, baseline subtracted and normalized to total ion current within a mass/charge (m/z) range of 1500 to 50000 Da.

### Phagocytosis

A clinical S. epidermidis strain was labelled with FITC by incubating 10⁹ bacteria from an exponential growth culture with 100 μg/ml FITC for 1 hour in 0.1 M carbonate buffer pH 9.6. Bacteria were washed twice with PBS, suspended in RPMI/HSA and stored at −20° C. Isolated human neutrophils or peripheral blood mononuclear cells (PBMN) at 5×10⁶ c/ml were mixed with FITC-labelled bacteria (ratio of 10 bacteria per phagocyte) and human serum or purified IgG in the presence or absence of inhibitor with a final volume of 50 μl.

Samples were incubated for 15 min at 37° C. in a round-bottom microplate under vigorous shaking (700 rpm on a microplate shaker). The phagocytosis reaction was terminated by the addition of 150 μl paraformaldehyde (1% final concentration) and samples were analysed for neutrophil associated fluorescence by flow cytometry. Sera used for opsonisation was a pool of 15 sera from healthy individuals stored at −80° C.

To eliminate the contribution of complement, the serum pool was heated for 30 min at 56° C. As an alternative for the role of IgG, purified human IgG for intravenous use was used (Sanquis, Amsterdam, The Netherlands).

### Cell Lines

A mouse macrophage (P388D1) and mouse B-lymphocyte (IIA1.6) cell line transfected with human FcγR (CD32a and CD64) were used in binding and phagocytosis experiments. Cells were maintained in RPMI containing 10% foetal calf serum and subcultured weekly. Cells were collected, washed once with RMPI/HSA, adjusted to 5×10⁶ cells/ml and used in phagocytosis experiments with human serum as described for isolated human neutrophils.

For inhibition of anti-mouse FcγR on P388D1 cells the PE-labelled anti-mouse FcγRII and III rat mAb (2.4G2) were used in the presence or absence of inhibitors.

### ELISA

Two different sets of ELISA experiments were performed using C-terminal His-tagged recombinant FcγR (FcγR-Ia, FcγR-IIa 131-His and 131-Arg variant, FcγR-IIb, and FcγR-IIIa 158-Val and 158-Phe variant) that were a generous gift from Prof. Jan van de Winkel (Genmab B. V., Utrecht, The Netherlands).

A) For the ligand inhibition ELISA, mAb anti-His (Research Diagnostics, Inc) coated ELISA plates (Greiner Bio-one) were incubated with optimal amounts of the various soluble FcγR, blocked with BSA and incubated with the inhibitors. Subsequently, a concentration range of HuMax-KLH (GenMab), optimised for each FcγR, was added followed by peroxidase labelled F(ab′)₂ goat anti-human IgG (F(ab′)₂ specific (Jackson ImmunoResearch Laboratories) and ABTS as substrate.

B) For the direct binding ELISA, the different inhibitors were coated at 1 μg/ml on ELISA plates, blocked with BSA and incubated with different concentrations His-tagged soluble FcγR. Binding was determined by incubation with peroxidase labelled mouse-anti-His (C-term; Invitrogen) antibody and ABTS substrate.

All ELISA assays used incubation steps of 75 minutes at room temp on a plate shaker at 300 rpm and 3 wash steps with PBS containing 0.05% Tween-20. Samples were diluted in PBS with Tween-20 and 0.2% BSA.

### Inhibition of Other FcγR

Purified recombinant inhibitors were tested for inhibition of different FcγR expressed on human leukocytes. Mononuclear cells were recovered from the Ficoll interface of heparinized blood. Cells were washed with RPMI/HSA, incubated with inhibitors and stained for anti-FcγR staining in combination with differently labelled specific markers.

Monocytes were identified by their forward and sideward scatter characteristics, B-lymphocytes were identified by scatters in combination with PE-labelled anti-CD19 (BD) staining and NK-cells were identified by scatters, APC-labelled anti-CD3 negative and PE-labelled anti CD16/CD56 (BD).

Antibodies used for the different FcγR were: PE or FITC-labelled 10.1 for anti-CD64, FITC-labelled nkp15 anti-CD16a, PE or APC-labelled anti-CD32 and control IgG1 mAbs PE-labelled anti-CD44 (hyaladherin) and anti-CD35 (Complement Receptor-1).

### Results

### Screening for CD32 Inhibition

To find potential inhibitors of human CD32, the FcγRIIa involved in phagocytosis of bacteria by leukocytes, inhibition of specific monoclonal antibody binding was used. Therefore a mAb was chosen that blocks functional activity of FcγRIIa, clone 7.3.

Several bacterial species were grown overnight and their cell-free supernatant collected to screen for inhibition of mAb staining of human neutrophils by flow cytometry. Supernatants recovered from Staphylococcus aureus gave the most consistent results with percentage inhibition ranging from 0% to 80% depending on the strain used (FIG. 22). Because S. aureus strain N315 supernatant was repeatedly effective with a strong inhibition and the genome of this strain was sequenced, N315 was chosen for further purification and identification of the CD32 inhibitory protein.

The inhibition was evident after 4 hours of bacterial culture, was stable at −20° C., retained in a 10,000 MW cut-off dialysis membrane and required only a short incubation time with the neutrophils. Ligand-dye affinity chromatography was used to enrich for activity by screening a panel of commercially available agarose-coupled dyes.

Reactive red 120 specifically retained activity that was eluted with 1 M NaCl. Elution fractions were screened for inhibition of the anti-CD32 neutrophil binding, either undiluted or 10-fold prediluted (FIG. 23A). Activity was found in a broad range of eluted fractions and the most active fractions were pooled and concentrated with a 10,000 MW cut-off device. This pooled fraction was separated into different fractions on a Sephadex-75 size-exclusion column. Again, fractions were screened for activity and peak fractions (around 15 kDa) pooled and concentrated (FIG. 23B). Analysis of TCA precipitated fractions with silver stained SDS-PAGE revealed still several different bands between 10 and 50 kDa.

As a final step in the purification, affinity chromatography was used with CD32 coated magnetic beads. Magnetic beads provide an efficient carrier with minimal death volume for convenient extraction of specific proteins from a small sample volume.

Commercially available His-tagged human CD32 was coupled to TALON-beads (covered with Cobalt that efficiently binds poly-histidines) and mixed with the enriched fraction from the Reactive red and Sephadex-75 columns. Beads were washed and associated proteins were dissolved in a small volume SDS-PAGE sample buffer for analysis on a silver stained 15% SDS-PAGE.

A band corresponding to the expected MW of this preparation of CD32 (32 kDa) was present along with a specific band of a proximally 12 kDa MW found only in the CD32 coated beads incubated with the enriched S. aureus fraction. This band was extracted, treated with trypsin and analysed for mass to identify the protein (FIG. 24D).

The sequence proved to be of one of the proteins of the invention, namely FLIPr. S. aureus strain N315 contains the gene for FLIPr that encodes a protein of 133 amino acids that contains a 28 amino acid leader peptide and a AXA cleavage site resulting in a mature 105 amino acid protein of 12.3 kDa.

As an alternative method for the identification of possible CD32 binding proteins in the enriched fraction, Ciphergen's SELDI-TOF approach was applied using IMAC30 and RS100 ProteinChip arrays. The IMAC30 array is an equivalent of the TALON magnetic beads and was loaded with Nickel to enable the binding of His-tagged CD32.

Alternatively, a RS100 array was used to couple CD32 using standard methodology and buffers onto the reactive surface. Both types of CD32-loaded arrays were incubated with the enriched S. aureus fraction, extensively washed, loaded with energy absorbing molecules and analysed in the ProteinChip machine for bound proteins.

Many mass peaks were found, mostly due to binding to the array itself, but a 12.3 kDa peak was clearly identified in the CD32 array only (FIG. 24C).

Searching the S. aureus sequenced genomes has resulted in the discovery of a homologous protein, called FLIPr-like (70% amino acid homology). This protein also inhibits the FPRL1 with a slightly better efficacy. Moreover, FLIPr-like also effectively inhibits the other receptor family member, the Formyl Peptide Receptor (FPR). FLIPr has limited activity towards the FPR and neither FLIPr nor FLIPr-like inhibits the third member of this receptor family, the FPRL2.

### Effects of Purified FLIPr and FLIPr-Like

Because FLIPr and FLIPr-like were expressed and purified as a recombinant protein in E. coli, direct verification of the proposed anti-CD32 activity was possible.

Human neutrophils were incubated with increasing concentrations FLIPr or FLIPr-like and checked for anti-CD32 staining. Both proteins inhibited concentration dependent mAb 7.3 binding to neutrophils. As a control, CHIPS did not affect neutrophil staining with mAb 7.3. A mutant of FLIPr-like that lacks the N-terminal 7 amino acids (FLIPr-like⁸⁻¹⁰⁴) retained comparable activity (FIG. 25). The inhibition of mAb 7.3 staining was concentration dependent for both FLIPr and FLIPr-like.

Direct binding of FLIPr and FLIPr-like to different recombinant soluble FcγRs was evaluated by ELISA. Therefore the proteins were coated onto microtiter plates and binding of FcγRs was detected using their His-tag. CHIPS coated plates served as control and showed no binding of any of the FcγRs tested. In general, FLIPr-like (FIG. 26B) was recognized by more FcγRs as compared to FLIPr (FIG. 26A).

For FLIPr the high (H131) affinity FcγRIIa and FcγRIIb were efficiently bound, while the low (R131) affinity FcγRIIa almost completely lost binding capacity. FcγRIa and IIIa did not bind to FLIPr but showed modest binding to FLIPr-like. The high affinity FcγRIIa and IIb bound very well to FLIPr-like. FLIPr and FLIPr-like directly bind to soluble FcγRs, as measured in a solid phase assay, and compete with mAb 7.3 for binding to an epitope involved in ligand binding.

Therefore, FLIPr and FLIPr-like were tested for direct inhibition of IgG ligand to immobilized FcγRs in an ELISA (FIG. 27). Both proteins efficiently prevented IgG (HuMax-KLH) binding to the FcγRIa and FcγRIIIa F158. Only FLIPR-like inhibited ligand binding to the high affinity (H131) FcγRIIa and IIb. For the low affinity (R131) FcγRIIa a modest inhibition by FLIPr was seen. FcγRIII was inhibited by FLIPr-like.

### Phagocytosis

A major function of FcγRs on neutrophils is the promotion of phagocytosis in conjunction with complement receptors. Therefore, FLIPr and FLIPr-like were tested for their ability to prevent phagocytosis of fluorescent-labelled Staphylococci by human neutrophils in the presence of human serum as IgG source.

Both proteins dose-dependently inhibited phagocytosis. FLIPr-like was more potent with 0.19 μg/ml as the minimal effective concentration (FIG. 28). FLIPr and FLIPr-like more efficiently inhibited lower amounts of heated serum that served as IgG source. CHIPS was used as control protein and consistently showed a small significant inhibition at concentrations of >1 μg/ml.

To eliminate other serum factors that contribute to phagocytosis purified human IgG for intravenous use was used to opsonize the bacteria. FIG. 29A shows that bacteria are efficiently taken up by the neutrophils and both FLIPr and FLIPr-like at 3 μg/ml completely block the phagocytosis. CHIPS did not affect the phagocytosis of bacteria opsonized with purified IgG in contrast to the heated serum.

To test the efficacy of FLIPr and FLIPr-like for murine FcγRs, the mouse macrophage P388D1 cell line was used with human IgG opsonized bacteria. As shown for human neutrophils, mouse phagocytes were inhibited by FLIPr and FLIPr-like as well (FIG. 29B). Also for the mouse phagocytes, CHIPS did not interfere with phagocytosis by human purified IgG. It should be noted that bacteria opsonized with heated human serum as IgG source were also taken up by P388D1 cells, but CHIPS did not show any inhibition in contrast to the human neutrophil mediated phagocytosis (data not shown). FLIPr and FLIPr-like also inhibited human peripheral blood monocytes mediated phagocytosis (FIG. 30).

FLIPr and FLIPr-like only partially inhibited phagocytosis when non-heated human serum was used for bacterial opsonization (FIG. 31) Under these conditions the phagocytosis process strongly depends on the contribution of complement.

1. A FPLR-1 inhibitor selected from the group consisting of:
a) a FLIPr having the amino acid sequence:
MKKNITKTIIASTVIAAGLLTQTNDAKAFFSYEWKGLEIAKNLADQAKKD
DERIDKLMKESDKNLTPYKAETVNDLYLIVKKLSQGDVKKAVVRIKDGGP
RDYYTFDLTRPLEENRKNIKVVKNGEIDSIYWD;
b) a FLIPr-like having the amino acid sequence:
MKKNITKTIIASTVIAAGLLTQTNDAKAFFSYEWKGLEIAKNLADQAKKD
DERADKLIKEADEKNEHYKGKTVEDLYVIAKKMGKGNTIAVVKIKDGGKN
GYYTFDITRPLEEHRKNIPVVKNGEIDSITWY;
c) fragments of a) or b) having FPLR-1 inhibitory activity;
d) homologues of a), b) or c) having FPLR-1 inhibitory activity; and
e) derivatives of a), b), c) or d) having FPLR-1 inhibitory activity.

2. The FPLR-1 inhibitor as claimed in claim 1, wherein the fragment is a fragment having the N-terminal part of the sequence given under a) or b), in particular the FLIPr-like⁸⁻¹⁰⁴ mutant.

3. The FLPR-1 inhibitor as claimed in claim 1, wherein the derivative is a functionally similar molecule that is a peptidomimetic version of one of the inhibitors listed under a), b), c) or d) of claim 1.

4. The FLPR-1 inhibitor as claimed in claim 1 for use as a medicament.

5. The FLPR-1 inhibitor as claimed in claim 4, for use in the inhibition of the formyl peptide receptor-like1 (FPRLI).

6. The FLPR-1 inhibitor as claimed in claim 1 for use in the treatment of inflammatory diseases.

7. The FLPR-1 inhibitor as claimed in claim 6, wherein the disease is caused by inflammatory reactions involving amyloids.

8. The FLPR-1 inhibitor as claimed in claim 1 for use in the treatment of neurodegenerative diseases.

9. The FLPR-1 inhibitor as claimed in claim 8, wherein the neurodegenerative disease is Alzheimer's disease.

10. The FLPR-1 inhibitor as claimed in claim 1 for use in the inhibition of the Fc-receptor.

11. The FLPR-1 inhibitor as claimed in claim 10, wherein the Fc-receptor is the Immunoglobulin G Fc Receptor II.

12. The FLPR-1 inhibitor as claimed in claim 1 for use in the treatment of immune complex-mediated diseases.

13. The FLPR-1 inhibitor as claimed in claim 12, wherein the immune complex-mediated diseases are autoimmune diseases.

14. A pharmaceutical composition, comprising a pharmaceutically acceptable excipient and a FLPR-1 inhibitor as claimed in claim 1.

15. A pharmaceutical composition as claimed in claim 14, wherein the composition is for use in medicine.

16. A pharmaceutical composition as claimed in claim 14, wherein the composition is for use in the treatment of inflammatory diseases.

17. A pharmaceutical composition as claimed in claim 16, wherein the disease is caused by inflammatory reactions involving amyloids.

18. A pharmaceutical composition as claimed in claim 14 for use in the treatment of neurodegenerative diseases.

19. A pharmaceutical composition as claimed in claim 18, wherein the neurodegenerative disease is Alzheimer's disease.

20. A pharmaceutical composition as claimed in claim 14 for use in the inhibition of the Fc-receptor.

21. A pharmaceutical composition as claimed in claim 20, wherein the Fc-receptor is the Immunoglobulin G Fc 5 Receptor II.

22. A pharmaceutical composition as claimed in claim 14 for use in the treatment of immune complex-mediated diseases.

23. A pharmaceutical composition as claimed in claim 22, wherein the immune complex-mediated diseases are autoimmune diseases.

24. Use of a FLPR-1 inhibitor as claimed in claim 1 for the preparation of a medicament for the treatment of inflammatory diseases.

25. The use as claimed in claim 24, wherein the disease is caused by inflammatory reactions involving amyloids.

26. The use as claimed in claim 25 for use in the treatment of neurodegenerative diseases.

27. The use as claimed in claim 26, wherein the neurodegenerative disease is Alzheimer's disease.

28. Use of a FLPR-1 inhibitor as claimed in claim 1 for the preparation of a medicament for the treatment of immune complex-mediated diseases.

29. The use as claimed in claim 22, wherein the immune complex-mediated diseases are autoimmune diseases.
